(19) United States
(12) Patent Application Publication
(10) Pub. No.: US 2003/0124568 A1
(43) Pub. Date: Jul. 3, 2003

(54) Novel central nervous protein, that modulates k+ flows

(76) Inventors: Daniela Spielvogel, Dossenheim (DE); Hans-Christian Kornau, Heidelberg (DE); Rohini Kuner, Heidelberg (DE); Gisela Eisenhardt, Heidelberg (DE); Annette Trutzel, Frankenthal (DE)

Correspondence Address: KEIL & WEINKAUF, 1350 CONNECTICUT AVENUE, N.W., WASHINGTON, DC, 20036, US

(21) Appl. No.: 10/203,821

(22) PCT Filed: Feb. 15, 2001
(86) PCT No.: PCT/EP01/01730

(51) Int. Cl. C12Q 1/68; G01N 33/53; A01K 67/00; C07H 21/04; C07K 14/47; C12P 21/02; C12N 5/06

(52) U.S. Cl. 435/6; 435/7.1; 435/69.1; 435/320.1; 435/325; 530/350; 530/388.1; 536/23.5; 800/8

(57) ABSTRACT

The present invention relates to nucleic acid sequences and isolated proteins which are novel interaction partners for inwardly rectifying potassium channels (Kirs), in particular G protein-coupled inwardly rectifying potassium channels (GIRKs). These interaction partners form protein complexes together with Kirs and GIRKs. The invention further relates to a charged domain of the novel interaction partners which binds to the complex intracellular region of Kirs and influences the activity of Kirs in general or of GIRKS in particular. The invention additionally relates to protein complexes composed of interaction partners and inwardly rectifying potassium channels, nucleic acid sequences or recombinant nucleic acid constructs which code for such proteins or domains, and the uses thereof. The invention additionally relates to protein complexes composed of the novel proteins with other proteins. The invention also relates to host organisms, specifically transgenic animals, which comprise the novel nucleic acid sequences or the recombinant nucleic acid constructs, and to mono- or polyclonal antibodies directed against the isolated proteins. The invention additionally relates to methods for discovering partners, that is to say low or high molecular weight substances which bind specifically to the novel interaction partners.

The present invention relates to nucleic ac sequences and isolated proteins which are novel interaction partners for inwardly rectifying potassium channels (Kirs), in particular G protein-coupled inwardly rectifying potassium channels (GIRKs). These interaction partners form protein complexes together with Kirs and GIRKs. The invention further relates to a charged domain of the novel interaction partners which binds to the complex intracellular region of Kirs and influences the activity of Kirs in general or of GIRKs in particular. The invention additionally relates to protein complexes composed of interaction partners and inwardly rectifying potassium channels, nucleic acid sequences or recombinant nucleic acid constructs which code for such proteins or domains, and the uses thereof. The invention additionally relates to protein complexes composed of the novel proteins with other proteins. The invention also relates to host organisms, specifically transgenic animals which contain the novel nucleic acid sequences or the recombinant nucleic acid constructs, and to mono- or polyclonal antibodies directed against the isolated proteins. The invention additionally relates to methods for discovering partners, that is to say low or high molecular weight substances which bind specifically to the novel interaction partners.

In the vertebrate central nervous system, G protein-coupled inwardly rectifying potassium channels (GIRKs) are involved in regulating the excitability of neurons by increasing the potassium conductivity of the cell membrane. The first cloning of a GIRK subunit (GIRK1) took place in 1993 (Kubo et al., Nature, 364, 1993:802-806; Dascal et al., Proc. Natl. Acad. Sci., USA, 90, 1993:10235-10239). To date, 4 sequence-related GIRK subunits have been described (GIRK2-5), which form heteromeric, tetrameric channels in the ratio 2:2 with GIRK1 (Kofuji et al., Proc. Natl. Acad. Sci., USA, 92, 1995:6542-6546; Yang et al., Neuron, Dec. 15, 6, 1995:1441-1447; Tucker et al., J. Biol., Chem., 274, 47, 1999:33393-33397); expression of the channels has been detected not only in neurons but also in atrial and endocrine cells (Karschin et al., FEBS Lett., 348, 2, 1994:139-144).

In the central nervous system, Gi/Go-coupled receptors trigger the activity of GIRK channels. In the presence of an agonist they catalyze the release of the β,γ subunit of trimeric G proteins; the β,γ subunit binds directly to the GIRK complex, thus stabilizing the interaction with phosphatidylinositol 4,5-bisphosphate, which results in enhanced activation of the GIRK channel (Huang et al., Nature, 391, 1998:803-806). Various neurotransmitters (e.g. adenosine, GABA or serotonin) trigger GIRK currents in this way, in order to modulate directly the electrical properties of the dendrites (Takigawa and Alzheimer; J. Physiol.[Lond], 517, Pt2, 1999:385-390). GABA_{B} receptors belong to the group of receptors which display their effect via GIRKs. They are involved in changes in synaptic efficiency, which forms the basis of learning and memory processes. GABA_{B} receptor agonists show beneficial effects in animal models of chronic pain and cocaine dependence. Antagonists have beneficial effects in models of absence epilepsy (Bettler et al., Curr. Opin. Neurobiol., 1998:345-350). Activation of GABA_{B} receptors suppresses overexcited neuronal linkages since they cause the opening of GIRK channels. Molecular targets by means of which it is possible to influence the GIRK currents are suitable for the treatment of epilepsy, stroke, cognitive losses, chronic pain and other neurological disorders, and for the treatment of psychological disorders such as anxiety, depressive disorders, schizophrenia, migraine and others. Evidence that such targets are also effective points of attack for treating alcohol dependence is shown by the fact that ethanol enhances the function of GIRKs coupled to GABA_{B} receptors in granule cells of the cerebellum (Lewohl et al., Nat. Neurosci. 2, 12, 1999:1084-1090; Kobayashi et al., 1999, Nat. Neurosci., 2, 12, 1999:1091-1096).

In animal models, the connection between GIRK channels and convulsions like those occurring in epileptic seizures has already been shown: null mutations for a GIRK subunit (GIRK2) in transgenic mice lead to spontaneous convulsions, an increased sensitivity to pharmacologically induced seizures, and a reduced amount of mRNA for the GIRK1 subunit (Signorini et al., Proc. Natl. Acad. Sci., USA, 94, 3, 1997:923-927). In an animal experimental model of convulsions, in which motor seizures are induced by electrical stimulation of the brain, it is likewise possible to detect changes in the expression pattern of GIRK1 and 2 in dentate gyrus cells (Pei et al., Neuroscience, 90, 2, 1999:621-627). GIRK currents evidently play a crucial part in many physiological relationships. Regulation of the number of GIRK proteins in the membrane and modulation of the currents through a GIRK channel is of great importance in this connection.

Interaction partners of GIRKs having an effect on the K⁺ currents are accordingly potential targets for the treatment of neurological disorders such as epilepsy. GIRK1 on its own has only low activity, but it differs from the other GIRK subunits in that it can associate with other family members (GIRK2-4) and thus may enhance their activity and alter their single channel characteristics (Chan et al., J. Biol. Chem., 272, 10, 1997:6548-6555). GIRK1 thus differs from the other GIRK subunits. Proteins which interact with GIRK1 might have a direct effect on the conductivity or the opening time of the channels or on the number of channels capable of functioning in the membrane.

GIRKs are also expressed in the heart. Their function there is to activate the K⁺ conductivity of atrial cells and thus reduce the heart rate (Kobo et al., Nature, 364, 1993:802-806).

Since GIRKs play a central part in various pathological processes in the central nervous system and the heart or are involved in processes of this type, they and their interaction partners with or without regulatory functions are sought-after targets for developing new drugs.

It is an object of the present invention to identify and characterize novel proteins which interact with the GIRKs, and to make it possible to develop molecular test systems with which many thousands of different compounds can be screened for high-affinity substances within a short time.

The object on which the present invention is based is accordingly achieved by the subject matter of the claims described herein.

We have found that this object is achieved by the novel isolated nucleic acid sequence selected from the group:

a) of a nucleic acid sequence having the sequence depicted in SEQ ID NO: 1, SEQ ID NO: 3; SEQ ID NO: 5 or SEQ ID NO: 7,

b) nucleic acid sequences which are derived as a result of the degeneracy of the genetic code from the nucleic acid sequence depicted in SEQ ID NO: 1, SEQ ID NO: 3; SEQ ID NO: 5 or SEQ ID NO: 7,

c) derivatives of the nucleic acid sequence depicted in SEQ ID NO: 1 or SEQ ID NO: 3, which code for polypeptides having the amino acid sequences depicted in SEQ ID NO: 2 or SEQ ID NO: 4 and have at least 60% homology at the amino acid level, with negligible reduction in the biological activity of the polypeptides,

d) equivalents of the sequences specified under (a) to (c) which still have biological activity.

It has been found that the proteins encoded by the novel nucleotide sequences interact with GIRK1.

Accordingly, the novel proteins having the amino acid sequences depicted in SEQ ID NO: 2 or SEQ ID NO: 4, and the functional derivatives, analogs and equivalents thereof, are novel interaction partners of Kirs, specifically of GIRKs. These interaction partners are referred to hereinafter as Mogli proteins or Mogli for short.

The novel proteins are able to form complexes with functional GIRK channels and thus also for example to change the K⁺ currents through the GIRK channels. This indicates that the novel proteins are modulators of the GIRK-mediated currents.

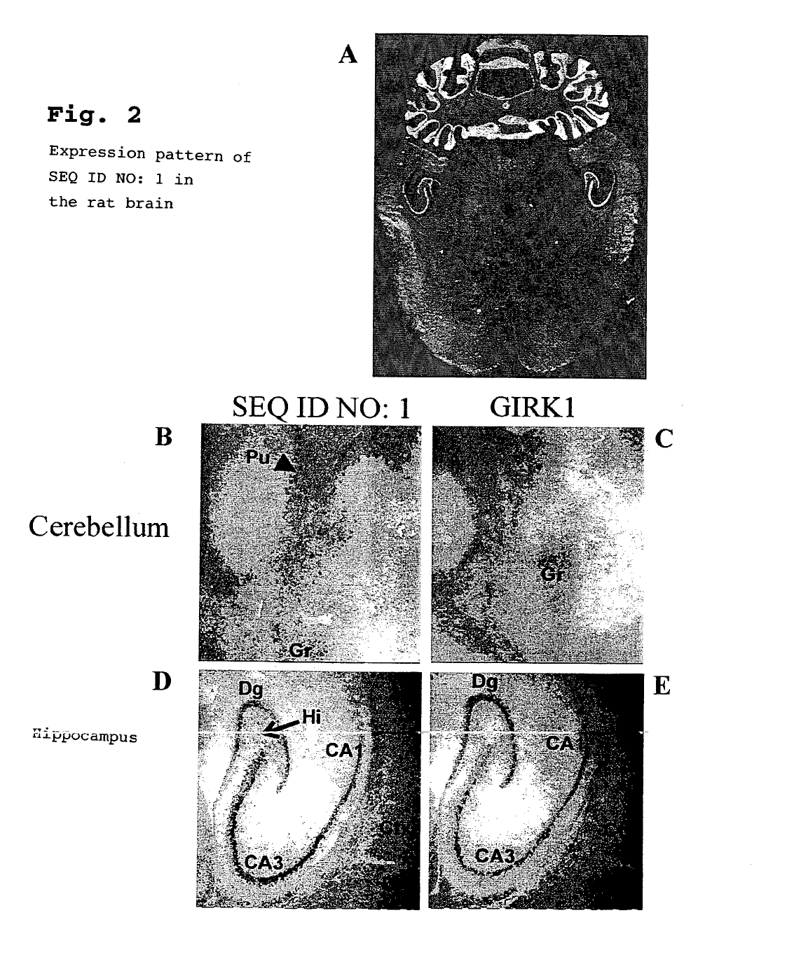

In situ hybridization with a novel nucleic acid sequence or parts thereof reveal a strong expression of an mRNA coding for the novel protein in the hippocampus, cortex, cerebellum, especially also Purkinje cells, and a lower expression in thalamic nuclei, striatum and the nucleus of the midbrain and brainstem (see FIG. 2 and example 4).

It has additionally been found that public databases contain a cDNA (AB002372) whose nucleotide sequence is 60% identical over a length of 1400 nt (=nucleotides) with the novel sequence SEQ ID NO: 3. This sequence was derived from a project in which a hundred cDNA fragments as large as possible from the brain were translated in vitro and, when proteins larger than 60 kDa were obtained, the relevant cDNA was sequenced (KiAA0374 Nagase et al.; DNA Res. 4:141-150, 1997). The publication indicates that there is very weak expression of this related mRNA in the brain. No further functional description of this mRNA or of a protein encoded by the mRNA is to be found in the publication. The sequence is 43% identical at the amino acid level with SEQ ID NO: 4 over a length of 473 amino acids. It can therefore be assumed that a related molecule is concerned in this case.

The protein (KIAA0374), deposited under ABOO2372 or AF187733 in the EMBL database has recently been assigned a regulatory function during transmitter release (Lao et al., Neuron, 25 2000:191-201). The protein called syntaphilin binds via a charged domain (“coiled-coil” or “cc”) to syntaxin-1, an essential constituent of the SNARE complex, or more accurately of the core complex consisting of syntaxin-1, SNAP-25 and VAMP, the formation of which is necessary for vesicular release of transmitters. This inhibits formation of the core complex, and transmitter release is reduced (Lao et al., Neuron, 25 2000:191-201). The amount of active synaptic syntaphilin thus regulates the efficiency of vesicle exocytosis, a variable which is important for many neurological disorders. The expression of syntaphilin is rather weak compared with syntaxin-1, so that it can inhibit only the formation of a limited part of the complete SNARE complexes. The term SNARE complex also means those proteins which interact with the core complex, in particular regulate it, such as, for example, the proteins listed hereinafter. The novel proteins might therefore likewise be involved in the regulation of transmitter release. This function can be inferred for example from the similarity of the sequence (SEQ ID NO: 8) of the novel protein of “cc” domain of syntaphilin. Syntaphilin is expressed in the cortex, hippocampus, olfactory bulb, striatum, midbrain and pons (corpus callosum), and thus differently from Mogli.

Further interaction partners of Mogli have been found in a yeast two-hybrid system using the N terminus of the novel protein (see example 7). These are preferably proteins encoded by those with KIAA0622 (ABO14522) and KIAA0627 (AB014527), or proteins of the EB family. The binding of these further interaction partners may depend on previous binding of proteins to Mogli or take place independently of this binding, or else be blocked or else promoted by previous binding of proteins to Mogli. Functions other than those mentioned above by way of example may be mediated by this interaction, like, for example, an intervention in the regulation of the cell cycle is possible through an interaction with the protein EB3 (ABO25186) (Nakagawa et al., Oncogene, 2000; 19(2); 210-216; Nakagawa et al., Cancer Res., 2000; 60(1); 101-105).

The term “protein complex or complex” is intended to mean hereinafter the protein complexes composed of at least one novel protein and at least one other protein, such as the GIRK complex, the SNARE complex or other complexes.

The term “biological activity” means according to the invention that a protein comprises at least one, preferably more than one of the biological activities possessed by a protein encoded by the novel nucleic acid sequence. Accordingly, an essential biological activity can be understood to mean that a novel protein interacts with a GIRK protein, in particular in a two-hybrid screen. An exemplary method with which the interaction can be determined is described in the examples. It was found in this way that an essential part in the interaction with GIRK is played in particular by the domains shown in SEQ ID NO. 6 and 8 (rat/human).

Biological activity accordingly also means that the protein specifically interacts with one of the antibodies described below, i.e. that it has epitopes which are recognized by antibodies which specifically bind to a protein or protein fragment encoded by SEQ ID NO. 1, 3, 6 or 8. “Specifically” means in particular that these antibodies interact only very weakly or not at all with syntaphilin. The skilled worker is aware of methods and tests with which such antibodies can be produced and tested (see below).

The novel protein preferably interacts with GIRK1 and/or proteins which are involved in transmitter release, in particular SNARE complexes and associated proteins, and the aforementioned other protein complexes. The novel protein is able to influence, through the interaction with GIRK1 or other KIR, the ion conductivity, specifically the K⁺ conductivity, of novel protein complexes.

“Biological activity” also means according to the invention that the protein may be involved in the regulation of transmitter release. A method for testing this regulation is described, for example, in Lao et al., Neuron, 25, 2000:191-201. Accordingly, biological activity also encompasses binding of the novel protein with a protein of the SNARE complex or with a protein associated therewith. This may inhibit or activate the formation of the SNARE complex. Examples of proteins which interact with the SNARE complex are MUNC18, N-Sec1, rbSec1, complexin, Doc2, tomosyn, NSF, snapin, the septin CDCrel-1 and SNAPs.

The essential biological property of the novel proteins (=Mogli), in particular their charged domain which mediates the binding to GIRK1, and of the novel protein complexes means the properties of the transmembrane region(s), of the amino-terminal region and of the carboxy-terminal region of the protein alone or in the protein complex. These protein regions make the specific biological effect of the proteins or protein complexes possible. These essential biological properties additionally comprise high-affinity binding (Kd<10 nM) of specific synthetic or natural molecules to the novel proteins having the amino acid sequence depicted in SEQ ID NO: 2 or SEQ ID NO: 4, and the interaction with the abovementioned, known proteins, e.g. to give the novel protein complexes. The interaction influences, as an essential biological property, the ion conductivity, specifically the K+ conductivity, of the complexes or the regulation of transmitter release and the activity of the SNARE complexes.

The novel isolated proteins mean proteins which comprise an amino acid sequence depicted in SEQ ID NO: 2 or SEQ ID NO: 4, or a sequence obtainable therefrom by substitution, inversion, insertion or deletion of one or more amino acid residues, where at least one of the essential biological properties of the protein depicted in SEQ ID NO: 2 or SEQ ID NO: 4 is still retained. This may entail, for example, particular amino acids being replaced by those with similar physicochemical properties (bulk, basicity, hydrophobicity etc.). Examples of replacements are arginine residues by lysine residues, valine residues by isoleucine residues or aspartic acid residues by glutamic acid residues. However, it is also possible for one or more amino acids to be transposed, added or deleted in their sequence, or a plurality of these measures can be combined together. The proteins which have been modified in this way by comparison with SEQ ID NO: 2 or SEQ ID NO: 4 have at least 60%, preferably at least 70%, and particularly preferably at least 90%, sequence identity over the entire length of the sequence with the sequences SEQ ID NO: 2 or SEQ ID NO: 4, calculated by the algorithm of Altschul et al., J. Mol. Biol., 215, 403-410, 1990. The identity with SEQ ID NO: 6 or 8 is at least 75%, preferably 80%, particularly preferably 85%, very particularly preferably 90% or more.

These proteins encoded by the abovementioned nucleic acids are present in the novel protein complexes. The novel protein complexes comprise at least one protein such as, for example, a Kir or a SNARE complex protein, and at least one novel protein having the amino acid sequence depicted in SEQ ID NO: 2 or SEQ ID NO: 4, or a sequence obtainable therefrom by substitution, inversion, insertion or deletion of one or more amino acid residues, wherein at least one of the essential biological properties of the protein depicted in SEQ ID NO: 2 or SEQ ID NO: 4 or of the protein complex is still retained. In the protein complexes, the protein encoded by the novel nucleic acid sequence influences, for example, the K⁺ conductivity. Thus it modulates for example advantageously the K⁺ conductivity of the protein or of the complex. The Kir proteins are advantageously so-called GIRK proteins. It advantageously modulates transmitter release.

The novel protein complexes mean Kir channels, advantageously GIRK channels, which contain a GIRK1 subunit, and at least one protein having the amino acid sequence depicted in SEQ ID NO: 2 or SEQ ID NO: 4. The article “Primary structure and functional expression of a rat G-Protein-coupled muscarinic potassium channel” (Kubo et al.; Nature Aug. 26, 1993, Vol. 364, pp. 802-806) described for the first time suitable GIRK1 subunits which may advantageously be present in the protein complexes. The described clone was called GIRK1 (database entry: 15-OCT-1993; ID: RNGIRK1A; AC: L25264). Entry of the sequence of the human homolog in the public databases (ID: HS07918; AC: U07918) took place on 10-APR-1994.

Analysis of the novel amino acid sequence using the PROSITE pattern search revealed a number of possible phosphorylation sites on the protein. Thus, for example, possible phosphorylation sites were found for a cAMP- and cGMP-dependent protein kinase, a protein kinase C or a casein kinase II. It is to be assumed that the protein is subject to regulation by phosphatases or kinases. In particular, phosphorylation sites located in the region of the conserved charged domain (see SEQ ID NO: 6 and SEQ ID NO: 8) possibly have a direct influence after (de)phosphorylation on the interaction of Mogli with other proteins, e.g. with GIRK1 or with proteins involved in transmitter release, e.g. SNARE complexes.

The interaction with GIRK1 is mediated by three domains: the intracellular amino terminus and carboxyl terminus of GIRK1, and a charged, α-helical (based on secondary structure predictions) domain in the novel protein.

One aspect of the invention is the complex of these interacting domains, and the Mogli domain which is involved in the interaction.

The domains responsible for the interaction in the protein complex have been analyzed through deletion constructs and subsequent analysis in the two-hybrid system (Fields et al., TIgS Vol. 10, 8, 1994:286-292 and Nature, Vol. 340, 1989:245-246, Chien et al., Proc. Natl. Acad. Sci. USA, vol. 88, 1991, 9578-9582). SEQ ID NO: 5 describes the rat domain and SEQ ID NO: 7 describes the human domain. The sequences SEQ ID NO: 6 and SEQ ID NO: 8 represent the corresponding protein sequences.

The isolated protein and its functional variants can advantageously be isolated from the brain of mammals such as Homo sapiens or Rattus norvegicus. Functional variants are to be understood to include homologs from other mammals.

A further aspect of the invention are nucleic acid sequences which code for the proteins described above, in particular for those having the primary structure depicted in SEQ ID NO: 2 or SEQ ID NO: 4. The nucleic acid sequence from Rattus norvegicus or Homo sapiens is depicted in SEQ ID NO: 1 or in SEQ ID NO: 3.

After isolation and sequencing it is possible to obtain the novel nucleotide sequences SEQ ID No: 1 and SEQ ID No: 3 or their functional equivalents such as, for example, allelic variants. Allelic variants mean variants of SEQ ID No: 1 or SEQ ID No: 3 which display 70 to 100% homology at the amino acid level, preferably 80 to 100%, very particularly preferably 90 to 100%. The homologies may advantageously be higher over some regions of the sequences. The novel nucleotide sequences SEQ ID No: 1 and SEQ ID No: 3 or their functional equivalents display at the DNA level a homology of at least 65%, preferably of at least 75%, particularly preferably of at least 85%, very particularly preferably of at least 90%, over the entire DNA region indicated in SEQ ID NO: 1 and SEQ ID NO: 3.

Allelic variants include, in particular, those functional variants which are obtainable by deletion, insertion or substitution of nucleotides from the sequence depicted in SEQ ID NO: 1 or SEQ ID NO: 3, where at least one of the essential biological properties is still retained. Novel proteins in which an essential biological property is still present advantageously mean proteins which still have at least 20%, preferably 50%, particularly preferably 75%, very particularly preferably 90% of the biological activity, for example in relation to increasing the K⁺ conductivity compared with the initial protein. Homologs or sequence-related nucleic acid sequences can be isolated from all mammalian species, including humans, by conventional methods by homology screening through hybridization with a sample of the novel nucleic acid sequences or parts thereof.

Functional equivalents also mean homologs of SEQ ID NO: 1 or SEQ ID NO: 3, for example their homologs from other mammals, truncated sequences, single-stranded DNA or RNA of the coding and noncoding DNA sequence.

Such functional equivalents can be isolated from other vertebrates such as mammals starting from the DNA sequences described in SEQ ID No: 1 or SEQ ID No: 3, or parts of these sequences, for example by conventional hybridization methods or the PCR technique. These DNA sequences hybridize under standard conditions with the novel sequences. It is advantageous to use for the hybridization short oligonucleotides from the conserved regions, advantageously from the interacting domain, for example from the charged regions or from the carboxy-terminal region, and these can be identified in a manner known to the skilled worker by comparisons with other proteins. However, it is also possible to use longer fragments of the novel nucleic acids or the complete sequences for the hybridization. These standard conditions vary depending on the nucleic acid used, whether oligonucleotide, longer fragment or complete sequence, or depending on which type of nucleic acid, DNA or RNA, is used for the hybridization. Thus, for example, the melting temperatures of DNA:DNA hybrids are about 10° C. lower than those of DNA:RNA hybrids of the same length.

Standard conditions mean, for example depending on the nucleic acid, temperatures between 42 and 58° C. in an aqueous buffer solution with a concentration between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids advantageously comprise 0.1×SSC and temperatures between about 20° C. and 45° C., preferably between about 30° C. and 45° C. The hybridization conditions for DNA:RNA hybrids advantageously comprise 0.1×SSC and temperatures between about 30° C. and 55° C., preferably between about 45° C. and 55° C. These temperatures stated for the hybridization are melting temperatures calculated by way of example for a nucleic acid with a length of about 100 nucleotides and a G+C content of 50% in the absence of formamide. The experimental conditions for the DNA hybridization are described in relevant textbooks of genetics such as, for example, Sambrook et al., “Molecular Cloning”, Cold Spring Harbor Laboratory, 1989, and can be calculated by formulae known to the skilled worker, for example depending on the length of the nucleic acids, the nature of the hybrids or the G+C content. The skilled worker can find further information on hybridization in the following textbooks: Ausubel et al. (eds), 1998, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Homologs of the sequences SEQ ID No: 1 and SEQ ID No: 3 additionally mean derivatives such as, for example, promoter variants. The promoters which precede the stated nucleotide sequences, together or singly, can be modified by one or more nucleotide exchanges, by insertion(s) and/or deletion(s) without, however, adversely affecting the functionality or effectiveness of the promoters. The promoters may moreover have their effectiveness increased by modifying their sequence or be completely replaced by more effective promoters even from organisms of different species.

The novel nucleic acid sequence also includes fragments of sequences SEQ ID NO: 1 and 3, includes in particular nucleic acid sequences which comprise a fragment of SEQ ID NO. 1 or 3 and which code for a polypeptide having at least one of the biological activities described above. Such a fragment preferably has the sequence of SEQ ID NO. 7 or 9 or of a homolog thereof which is at least 70% identical to SEQ ID No. 7 or 9. The sequence is preferably more than 80%, 90% and more preferably more than 95% identical. The encoded sequence may be responsible for the interaction of the novel protein with another protein, e.g. a KIR protein, in particular GIRK proteins, e.g. GIRK1, or with a protein involved in transmitter release, e.g. with proteins of the SNARE complex.

Derivatives also advantageously mean variants whose nucleotide sequence in the region from −1 to −1000 in front of the start codon has been modified in such a way that gene expression and/or protein expression is altered, preferably increased. Moreover, derivatives also mean variants which have been modified at the 3′ end. These alterations advantageously made at the 3′ end relate, for example, to terminators or sequences which have a beneficial effect on translation and/or transcription.

For optimal expression of heterologous genes in organisms, it is advantageous to modify the nucleic acid sequences to accord with the codon usage specifically used in the organism. The codon usage can easily be established on the basis of computer analyses of other known genes in the relevant organism.

It is additionally advantageous for the novel nucleic acids of SEQ ID NO: 1 or SEQ ID NO: 3 alone or the nucleic acids of SEQ ID NO: 1 or SEQ ID NO: 3 and one or more sequences which code, for example, for complexes such as GIRK or Kir proteins or for proteins involved in transmitter release, e.g. SNARE complexes or proteins interacting with the complexes, to be functionally linked to at least one genetic regulatory element to give the novel recombinant nucleic acid constructs.

The novel nucleic acid sequences are normally for this purpose functionally linked to genetic regulatory elements such as transcription and translation signals. This linkage may, depending on the required use, lead to an increase or decrease in gene expression. Host organisms are then transformed with the recombinant nucleic acid constructs produced in this way. In addition to these novel regulatory sequences, it is also possible for the natural regulation of these sequences to be present in front of the actual structural genes and, where appropriate, to have been genetically modified so that the natural regulation is switched off and the expression of the genes has been increased. The gene construct may, however, also have a simpler structure, that is to say no additional regulatory signals are inserted in front of the sequences, and the natural promoter with its regulation is not deleted. Instead, the natural regulatory sequence is mutated in such a way that the regulation no longer takes place, and gene expression is increased. It is also possible to insert additional advantageous regulatory elements at the 3′ end of the nucleic acid sequences. The nucleic acid sequences for sequences SEQ ID No: 1 or SEQ ID No: 3 may be present in one or more copies in the gene construct, or be located on separate gene constructs.

Advantageous regulatory sequences for the novel method are, for example, present in promoters such as the cos, tac, trp, tet, trp-tet, lpp, lac, lpp-lac, laciq, T7, T5, T3, gal, trc, ara, SP6, λ-PR or λ-PL promoter, which are advantageously used in Gram-negative bacteria. Further advantageous regulatory sequences are, for example, in the Gram-positive promoters such as amy and SP02, in the yeast promoters such as ADC1, MFα, AC, P-60, CYC1, GAPDH or in mammalian promotors such as CaM kinaseII, CMV, nestin, L7, BDNF, NF, MBP, NSE, β-globin, GFAP, GAP43, tyrosine hydroxylase, kainate receptor subunit 1, glutamate receptor subunit B.

It is possible in principle for all natural promoters with their regulatory sequences like those mentioned above to be used. It is also possible in addition advantageously to use synthetic promoters.

These regulatory sequences are intended to make specific expression of the nucleic acid sequences and protein expression possible. This may mean, for example, depending on the host organism, that the gene is expressed or overexpressed only after induction or that it is immediately expressed and/or overexpressed.

The regulatory sequences or factors may moreover preferably influence positively, and thus increase, the expression. Thus, enhancement of the regulatory elements can take place advantageously at the level of transcription, by using strong transcription signals such as promoters and/or enhancers. However, it is also possible in addition to enhance translation by, for example, improving the stability of the mRNA.

Enhancers mean, for example, DNA sequences which bring about increased expression via an improved interaction between RNA polymerase and DNA. Further regulatory sequences which may be mentioned by way of example are the locus control regions, silencers or respective part-sequences. These sequences can be used advantageously for tissue-specific expression.

A preferred embodiment is linkage of the novel nucleic acid sequence to a promoter, the location of the promoter being 5′ upstream. Further regulatory signals, such as 3′-located terminators or polyadenylation signals or enhancers can be used functionally in the nucleic acid construct and thus influence its expression.

The concept of the novel “recombinant nucleic acid construct or gene construct” also means complete vector constructs. These vector constructs or vectors are used for expression in a suitable host organism. It is advantageous for the novel nucleic acids and/or the genes which encode for example, Mogli, or complexes such as SNARE complex proteins and/or GIRKs to be inserted into a host-specific vector which makes optimal expression of the genes in the selected host possible. Vectors are well known to the skilled worker and can be found, for example, in the book Cloning Vectors (Eds. Pouwels P. H. et al. Elsevier, Amsterdam-New York-Oxford, 1985, ISBN 0 444 904018). Apart from plasmids, vectors also mean all other vectors known to the skilled worker, such as, for example, phages, viruses such as SV40, CMV, baculovirus, adenovirus, transposons, IS elements, phasmids, phagemids, cosmids, linear or circular DNA. These vectors may undergo autonomous replication in the host organism or chromosomal replication. Linear DNA is advantageously used for integration in mammals.

Expression of the novel nucleic acid sequences or of the recombinant nucleic acid construct can advantageously be increased by increasing the gene copy number and/or by strengthening regulatory factors which have a beneficial effect on gene expression. Thus, strengthening of regulatory elements can preferably take place at the level of transcription by using stronger transcription signals such as promoters and enhancers. However, it is also possible besides this to strengthen translation by, for example, improving the stability of the mRNA or increasing the reading efficiency of this mRNA at the ribosomes.

To increase the gene copy number, the nucleic acid sequences or homologous genes can be incorporated, for example, into a nucleic acid fragment or into a vector which preferably contains the regulatory gene sequences assigned to the particular genes, or promoter activity with an analogous effect. Regulatory sequences which strengthen gene expression are used in particular.

The novel nucleic acid sequences can be cloned together with the sequences coding for the GIRKs or sequences coding for proteins present in SNARE complexes or associated with them, such as, for example, those listed above, or having regulatory function, in a single vector and then be expressed in the required organism. An alternative possibility is also to put each of the described nucleic acid sequences and the sequences coding for said proteins, e.g. the GIRK proteins, into a single vector in each case and to introduce these separately into the particular organism by conventional methods such as transformation, transfection, transduction, electroporation or particle gun.

It is additionally possible for the novel nucleic acid construct or the novel nucleic acids also to be expressed in the form of therapeutically or diagnostically suitable fragments. To generate the recombinant protein it is possible to use vector systems or oligonucleotides which extend the nucleic acids or the nucleic acid construct by particular nucleotide sequences and thus code for modified polypeptides which simplify purification. Tags of this type disclosed in the literature are, for example, hexahistidine anchors or epitopes which can be recognized as antigens of various antibodies (Studier et al., Meth. Enzymol., 185, 1990:60-89 and Ausubel et al. [eds.], 1998, Current Protocols in Molecular Biology, John Wiley & Sons, New York).

Suitable host organisms are in principle all organisms which make it possible to express the novel nucleic acids, their allelic variants, their functional equivalents or derivatives or the recombinant nucleic acid construct alone or together with a sequence which codes for one of the abovementioned proteins, in particular a complex partner for forming complexes such as GIRK protein or SNARE protein. Host organisms mean, for example, bacteria, fungi, yeasts, or plant or animal cells. Preferred organisms are bacteria such as Escherichia coli, Streptomyces, Bacillus or Pseudomonas, eukaryotic microorganisms such as Saccharomyces cerevisiae, Aspergillus, higher eukaryotic cells from humans or animals, for example COS, Hela, HEK293, Sf9, CHO, PC12 cells or primary neuronal cell cultures.

If required, the gene product can also be expressed in transgenic organisms such as transgenic animals, for example mice, rats, sheep, cattle or pigs. Transgenic plants are also conceivable in principle. The transgenic organisms may also be so-called knockout animals.

It is moreover possible for the transgenic animals to contain a functional or nonfunctional novel nucleic acid sequence or a functional or nonfunctional nucleic acid construct alone or in combination with a functional or nonfunctional sequence which codes for Mogli or functional equivalents or derivatives.

A further novel embodiment of the transgenic animals described above are transgenic animals in whose germ cells or all or a part of the somatic cells, or in whose germ cells and all or a part of the somatic cells the novel nucleotide sequence has been modified by genetic engineering methods or interrupted by insertion of DNA elements.

The combination of the host organisms and the vectors appropriate for the organisms, such as plasmids, viruses or phages, such as, for example, plasmids with the RNA polymerase/promoter system, the phages λ, Mu or other temperate phages or transposons and/or other advantageous regulatory sequences forms an expression system. The term expression systems preferably means, for example, the combination of mammalian cells such as CHO cells and vectors such as pcDNA3neo vector or HEK293 cells and CMV vector, which are suitable for mammalian cells.

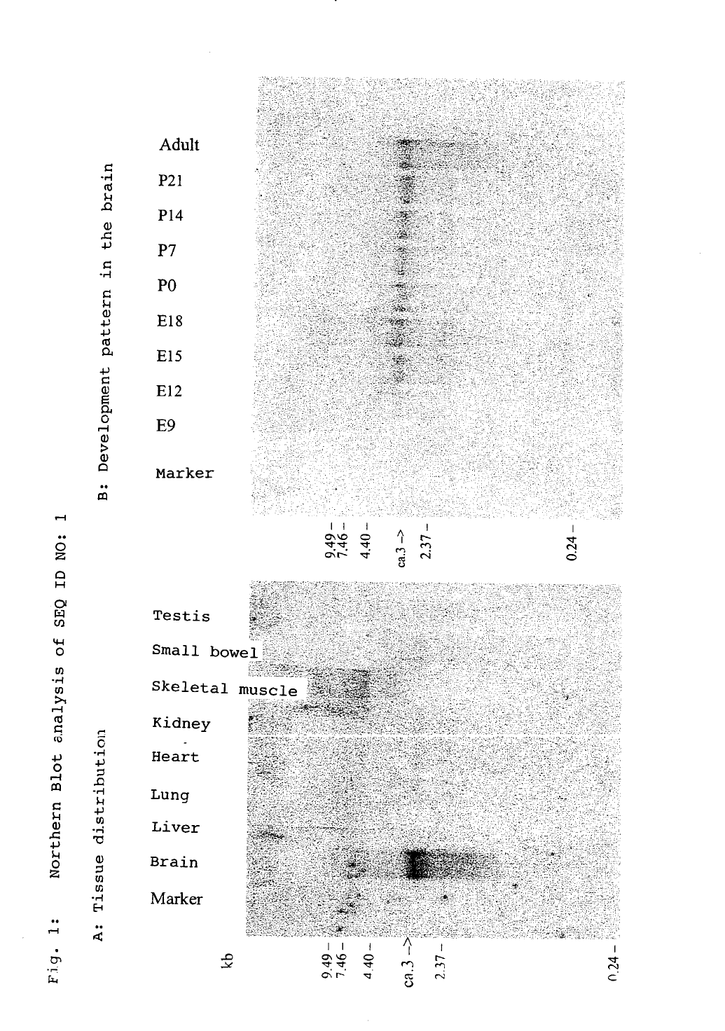

In situ hybridization with the novel nucleic acid sequence or parts thereof revealed strong expression in the hippocampus, cortex; cerebellum, especially also Purkinje cells, and lower expression in thalamic nuclei, striatum and the nuclei of the midbrain and brainstem (see FIG. 2 and Example 4). FIGS. 1 and 2 show the analysis of the expression of the mRNA corresponding to SEQ ID NO: 1. 1 shows the Northern blot, 2 the in situ hybridization (see Example 3 and 4).

The expression pattern of SEQ ID NO: 1 overlaps with that of GIRK1 and indicates an important CNS function of the protein depicted in SEQ ID NO: 2 or SEQ ID NO: 4. The mRNAs coding for SEQ ID NO: 2 and GIRK1 are coexpressed in most hippocampal neurons. In addition, SEQ ID NO: 1, but only a very little GIRK1, is expressed in inhibitory interneurons of the dentate gyrus. SEQ ID NO: 1 might keep the excitability of important hippocampal neurons, for example the pyramidal cells, in balance both via the interaction with GIRK1 and via other mechanisms as yet unknown.

The hippocampus is the crucial brain structure for storing new memory contents. A protein having the sequence SEQ ID NO: 2 is thus an interesting target for understanding in relation to learning and memory and for developing novel cognitive enhancers. As part of the limbic system, the hippocampus also influences moods and feelings. Drugs directed against SEQ ID NO: 2 or SEQ ID NO: 4 and their functional equivalents, homologs or derivatives thus represent potential antidepressants or anxiolytics and can be used for cognitive disorders. Finally, the hippocampus is extensively involved in temporal lobe epilepsies, which makes a protein having SEQ ID NO: 2 or SEQ ID NO: 4 an attractive target for novel medicines to counter this common disorder. There are regions in the cortex which integrate and process sensory information and convert it into suitable reactions. These sensory and motor centers are often also the starting points of epileptic seizures. Targeted influencing of the novel proteins or the novel protein complex might reduce the probability of convulsions in epileptics. The thalamic nuclei precede the cortex in sequence, and integrate the perceptions picked up by the sensory organs and transmit them to cortical structures. They are often the starting point of generalized fits. Strong expression of the novel proteins in the thalamic nuclei indicate that its activation or its inhibition may contribute to alleviation of fits in epileptics.

In contrast to GIRK1, SEQ ID NO: 1 is strongly expressed in the Purkinje cells of the cerebellum. SEQ ID NO: 2 might effectively influence the total activity of the cerebellum through modulation of the state of excitation of these cells. The cerebellar connections are crucially responsible for fine coordination of movements. Ataxias and other motor disorders such as, for example, dystonia might be based on deregulation of a protein having the novel sequence.

The basal ganglia, including the striatum, are important for the preparation, programming, initiation and termination of coordinated movements. Disturbances of muscle tone are caused in particular by an altered activity of the striatum.

The novel protein complexes or proteins thus represent interesting targets for developing novel substances which can be used to produce medicines for treating disorders such as neurological disorders such as epilepsy, stroke, psychological disorders such as anxiety, manic-depressive disorders, migraine, cognitive losses or movement disorders such as hypokinesia, hyperkinesia, dystonia, Parkinson's disease and other disturbances of muscle tone.

The novel protein shows an expression pattern which is similar but not identical to that of the closest known homolog (Lao et al., 2000). Mogli is, as already mentioned above, strongly expressed in the brain, in rats mainly weakly from the E12 stage onwards, strongly from the E15 onwards and increasingly up to the adult stage. Mogli is expressed particularly strongly in Purkinje cells and in granular cells of the cerebellum, likewise strongly in the cortex and in the hippocampus, more weakly in various thalamic nuclei, in the striatum and in the midbrain. Thus, in distribution and strength it shows expression which is very similar but by no means identical to that of syntaphilin (Lao et al., 2000). Since it presumably may be involved in transmitter release, therefore, the novel protein has an important role as point of attack for medicaments for neurological disorders such as, in particular, epilepsy, dystonia, stroke, cognitive losses, chronic pain and others, and the treatment of psychological disorders such as, in particular, anxiety, depressive disorders, schizophrenia, migraine and others. As regulators of synaptic transmitter release, the novel proteins represent particularly attractive points of attack for an effective pharmacotherapeutic intervention.

Database searches revealed that the gene for the novel protein is encoded in fragments on a BAC (BAC clone: KB1171G1; DT: 13-SEP-1999 ID: AP000427) from whose nucleic acid sequence data no protein sequence can be inferred. This BAC has been localized in the human genome and is located in the vicinity of the chromosomal locus which is associated with adult myoclonic epilepsy (Mikami et al., 1999). This correlation might also make a novel diagnostic method for this type of epilepsy possible. The nucleotide sequences SEQ ID NO: 1 and SEQ ID NO: 3 can be used for isolating and mapping genes for mRNAs which code for these nucleic acids or their functional equivalents, homologs or derivatives in the murine and in the human genome with conventional methods by homology screening, and for correlating with markers of human genetic diseases. This makes it possible to identify the gene causing particular genetic diseases, which considerably simplifies their diagnosis and makes new therapeutic approaches possible. It is thus possible with the aid of the nucleic acids as markers to diagnose genetic diseases.

The invention additionally relates to the use of the novel nucleic acids or parts thereof for gene therapy. Sequences complementary to the novel nucleic acids or parts thereof can also be used for gene therapy.

A further possibility for using the nucleotide sequence or parts thereof is to generate transgenic or knockout or conditional or region-specific knockout animals or specific mutations in genetically modified animals (Ausubel et al. [eds], 1998, Current Protocols in Molecular Biology, John Wiley & Sons, New York and Torres et al., [eds.], 1997, Laboratory protocols for conditional gene targeting, Oxford University Press, Oxford). It is possible by transgenic overexpression or genetic mutation (null mutation or specific deletions, insertions or modifications) through homologous recombination in embryonic stem cells to produce animal models which provide valuable further information about the (patho)physiology of the novel sequences alone or in a complex for example with the GIRKs or SNARE proteins. Animal models produced in this way may represent essential test systems for evaluating novel therapeutics which specifically influence the excitability of neurons.

Interaction of the known GIRK1 with the novel protein described in the invention, which was discovered using the two-hybrid system, possibly plays an important physiological role. This surprising finding makes novel exceptional treatments possible in relation to the abovementioned neurological and psychological disorders connected with GIRKs. Low molecular weight effectors or peptides which have a positive or negative effect on this interaction are agents which intervene in the K⁺ conductivity of membranes and thus can be used as a novel class of drugs. To date, no substances which influence the interaction between domains of Mogli and Kir or GIRK proteins and thus modulate the properties of these proteins have been described. It is likewise possible for low molecular weight affectors or peptides to influence the interaction of Mogli with proteins involved in transmitter release and thus regulate exocytosis. Use of the novel protein complex or of the novel proteins thus makes it possible to develop novel active ingredients or classes of active ingredients having a novel principle of action.

Use of the novel nucleic acid sequence, of the nucleic acid construct, of a novel protein complex or of the protein makes it possible to identify proteins which for example display specific binding affinities for the GIRK or SNARE protein complex or to identify nucleic acids which code for proteins which for example display specific binding affinities for the GIRK or SNARE protein complex or for the protein. It is advantageous to use for this purpose the two-hybrid system or other biochemical methods alone or in combination. It is possible in this way to determine intramolecular interaction domains of GIRKs and intermolecular interaction domains of complexes such as GIRKs or SNARE complexes and Mogli and thus pharmacotherapeutic intervention points.

One aspect of the invention is therefore the use of the two-hybrid system or biochemical methods for identifying the interaction domains of Moglis and their interaction partners and the use for pharmacotherapeutic intervention.

It is possible by analyses of the structure of the protein complex or of the novel protein specifically to find substances which display a specific binding affinity.

The described sequences SEQ ID NO: 1 and SEQ ID NO: 3 make it possible, with the aid of the two-hybrid system or other assays, to localize the amino acids responsible for the interaction and find substances with which it is possible to influence in particular the interaction between Mogli and GIRKs.

A further aspect of the invention relates to substances which specifically reduce or prevent the natural interaction of the GIRK1N terminus with the GIRK1C terminus or GIRK1 with the protein having SEQ ID NO: 2 or 4.

Substances of this type preferentially bind to the following sequence regions:

(i) to the amino acid sequence of GIRK1 or

(ii) to the amino acid sequence of SEQ ID NO: 2 or

(iii) to the amino acia sequence of SEQ ID NO: 4 or

(iv) to the interaction regions which are formed by the interacting domains of GIRK and SEQ ID NO: 2 or of GIRK and SEQ ID NO: 4, or

(v) to the interaction domains of SEQ ID NO: 2 or SEQ ID NO: 4 which are depicted in SEQ ID NO: 6 or SEQ ID NO: 8 respectively.

Besides substances which bind to these sequences, also suitable as substances which impede or prevent the interaction are these polypeptides themselves and parts of these polypeptides, in particular polypeptides which have a sequence of at least 5 amino acids of one of these sequences (i), (ii) and (iii).

A further aspect of the invention is a method for discovering substances with a specific binding affinity for the novel protein complex or protein, which comprises the following steps:

a) incubation of the protein(s) with the substance to be tested,

b) detection of the binding to the protein of the substance to be tested.

The detection of the binding takes place, for example, by measuring the activity of GIRKs, the change in the membrane potential or the K⁺ conductivity, or measuring transmitter release.

Systems for detecting the changes in properties of potassium channels may be designed as follows:

a) Ion-sensitive electrodes can be used to measure specifically changes in the potassium concentration in the environment or in the cells themselves caused by an altered K⁺ conductivity of the membrane (Uhlig et al., Anal. Chem., 69, 19, 1997:4032-4308)

b) Alterations in the membrane potential can be detected directly by specific incorporation of fluorophores into K⁺ channels, because these experience a change in conformation when the voltage changes (Mannuzzu et al., Science 271, 5246, 1996:213-216)

c) Changes in voltage at individual cells can be detected by suitable fluorescent dyes. Measurement systems of this type may be based on a single fluorophore indicator or on a two-component sensor which is based on a FRET (=fluorescence resonance energy transfer) effect (Gonzalez and Tsien, Chem. Biol., 4, 1997:269-277).

Systems for detecting the change in transmitter release may be designed as follows:

Cultivation of neurons as described by Bekkers et al. (Proc. Natl. Acad. Sci USA, 88, 1991:7834-7838) makes it possible to measure the autaptic response to an electrical stimulation. It is possible by means of transient expression of proteins as depicted in SEQ ID NO: 2, 4, 6 or 8 by viral systems (as described, for example, in Park et al., J. Neurosci, 17, 23, 1997:8975-8983) to measure changes in the response at autapses caused by the overexpressed protein and thus derive information about transmitter release (Lao et al., Neuron, 25, 2000:191-201).

Further embodiments of the invention are a method for discovering substances which inhibit or enhance the interaction of proteins having amino acid sequences like those depicted in SEQ ID NO: 2 and SEQ ID NO: 4 with complexes such as GIRKs or SNARE complexes. Interaction of proteins with the novel amino acids can be detected for example using the two-hybrid system, especially for GIRK1. It is also possible to carry out the methods by expressing the proteins in eukaryotic cells and linking with a reporter assay for activation of the GIRKs or the formation of SNARE complexes. This entails, for example, detection of the alteration in the membrane potential, the K⁺ conductivity or the transmitter release.

The protein activity of the proteins having the sequences SEQ ID NO: 2 or SEQ ID NO: 4 can be determined via antibodies. A further aspect of the invention is therefore a method for quantifying the protein activity of a protein having the sequences SEQ ID NO: 2 or SEQ ID NO: 4.

The regulatory sequences of the novel nucleic acids, in particular the promoter, the enhancers, locus control regions and silencers or part-sequences of each of them can be used for tissue-specific expression of this and other genes. This makes it possible to carry out brain-specific gene expression of nucleic acid constructs, specifically in the heart, hippocampus, cortex, cerebellum, in thalamic nuclei, in the striatum or the nuclei of midbrain and brainstem.

In order to isolate a DNA fragment which comprises the regions which regulate transcription of the sequences SEQ ID NO: 1 or SEQ. ID NO: 3, initially a genomic bank is screened with a cDNA probe positioned as far 5′ as possible. This is done by carrying out a homology search familiar to the skilled worker (Ausubel et al. [eds.], 1998, Current Protocols in Molecular Biology, John Wiley & Sons, New York). The transcription start on the isolated DNA fragment is then identified. The region in front of the transcription start is subsequently linked to a reporter gene such as β-galactosidase or GFP (=green fluorescent protein) and tested in cells or in transgenic animals such as mice to find whether it leads to the expression pattern specific for SEQ ID NO: 1 or SEQ ID NO: 3 (Ausubel et al., see above). The reporter gene can then be linked to other cDNAs in order to produce animal models in which the particular cDNA undergoes region-specific expression (see, for example, Oberdick et al., Science, 248, 1990:223-226).

Starting from the amino acid sequences SEQ ID NO: 2 or SEQ ID NO: 4 it is possible to generate synthetic peptides which are employed as antigens for producing antibodies. It is also possible to employ the polypeptide or fragments thereof for generating antibodies. Antibodies mean both polyclonal, monoclonal, human or humanized or recombinant antibodies or fragments thereof, single-chain antibodies or else synthetic antibodies. Novel antibodies or their fragments mean in principle all immunoglobulin classes such as IgM, IgG, IgD, IgE, IgA or their subclasses such as the subclasses of IgG or mixtures thereof. Preference is given to IgG and its subclasses such as, for example, IgG₁, IgG₂, IgG₂ₐ, IgG_{2b}, IgG₃ or IgG_{M}. Particular preference is given to the IgG subtypes IgG₁3/κ or IgG_{2b}/κ. Fragments which may be mentioned are all truncated or altered antibody fragments with one or two antigen-complementary binding sites, such as antibody parts with a binding site which corresponds to the antibody and is formed by a light and heavy chain, such as Fv, Fab or F(ab′)₂ fragments or single-chain fragments. Preference is given to truncated double-chain fragments such as Fv, Fab or F(ab′)₂. These fragments can be obtained, for example, by enzymatic means by eliminating the Fc part of the antibodies using enzymes such as papain or pepsin, by chemical oxidation or by genetic manipulation of the antibody genes. It is also possible and advantageous to use genetically manipulated non-truncated fragments. It is possible in particular to generate antibodies also for fragments of said sequences, e.g. of SEQ ID No. 6 or 8.

The antibodies or fragments can be used alone or in mixtures.

The antibody genes can be isolatd for the genetic manipulations in a manner known to the skilled worker, for example from the hybridoma cells (Harlow, E. and Lane, D. 1988, Antibodies: A Laboratory Manual, Cold Spring Harbor Press, N.Y.; Ausubel et al., [eds], 1998, Current Protocols in Molecular Biology, John Wiley & Sons, New York). This is done by culturing antibody-producing cells and isolating the mRNA from the cells at an adequate optical density of the cells by cell lysis with guanidinium thiocyanate, acidification with acetate buffer, extraction with phenol, chloroform/isoamyl alcohol, precipitation with isopropanol and washing with ethanol in a known manner. Subsequently, cDNA is synthesized from the mRNA with the aid of reverse transcriptase. The synthesized cDNA can be inserted directly or after genetic manipulation, for example by site-directed mutagenesis, introduction of insertions, inversions, deletions or base exchanges, into suitable animal, fungal, bacterial or viral vectors and be expressed in the appropriate host organisms. Preference is given to bacterial or yeast vectors such as pBR322, pUC18/19, pACYC184, lambda or yeast mu vectors for cloning the genes and expression in bacteria such as E. coli or in yeast such as Saccharomyces cerevisiae.

Specific antibodies against the novel proteins are suitable both as diagnostic reagents and as therapeutic agents for neurological or psychiatric syndromes.

It is also possible to use the cDNA, the genomic DNA, the regulatory elements of the novel nucleic acid sequences, as well as the polypeptide and fragments thereof in recombinant or nonrecombinant form for designing an assay system. This assay system is suitable for measuring the activity of the promoter or of the protein in the presence of the test substance. The methods of measurement involved here are preferably simple ones (calorimetric, luminometric, fluorescence-based or radioactive) permitting rapid measurement of a large number of test substances (Böhm, Klebe, Kubinyi, 1996, Wirkstoffdesign, Spektrum-Verlag, Heidelberg). The described assay systems permit screening of chemical libraries for substances which have measurable effects on SEQ ID NO: 2 or SEQ ID NO: 4 or the novel GIRK complex consisting of GIRK1 which has already been described and of the protein described in SEQ ID NO: 2 or SEQ ID NO: 4 or on the complexes between the novel protein and, for example, proteins involved in transmitter release. Identification of such substances represents the first step toward identifying novel medicines acting specifically on the K⁺ conductivity or transmitter release.

An alternative way of developing agents which act on the novel complex such as GIRK-protein complex or SNARE-protein complex consists of rational drug design (Böhm, Klebe, Kubinyi, 1996, Wirkstoffdesign, Spektrum-Verlag, Heidelberg). In this case, the structure or a part-structure of the protein depicted in SEQ ID NO: 2 or SEQ ID NO: 4, if available, or a model of the structure produced by computers, is used to find, with the assistance of molecular modeling programs, structures for which it is possible to predict a high affinity for Mogli. These substances are then synthesized and tested. High-affinity selective substances are tested for use as medicines for epilepsy, stroke and other neurological disorders.

Determination of the amount, activity and distribution of the novel protein, e.g. in a novel complex such as SNARE- or novel GIRK-protein complex, in particular of the protein depicted in SEQ ID NO: 2 or SEQ ID NO: 4 or of its underlying mRNA in the human body can be used for diagnosis of, detecting a predisposition to and monitoring of particular disorders. In the same way, the sequence of the cDNA of the sequences SEQ ID NO: 2 or SEQ ID NO: 4, and of the genomic DNA, can be used to make statements about genetic causes of and predispositions to particular disorders. It is possible to use for this purpose both DNA/RNA probes and antibodies of a wide variety of types. In these cases, the described nucleotide sequence SEQ ID NO: 1 or SEQ ID NO: 3 or parts thereof is used in the form of suitable probes for detecting point mutations or deletions/insertions/rearrangements.

The present nucleic acid sequence SEQ ID NO: 1 or SEQ ID NO: 3, its functional equivalents, homologs or derivatives, the protein encoded by it (SEQ ID NO: 2 or SEQ ID NO: 4) or the novel protein complex, and reagents derived therefrom (oligonucleotides, antibodies, peptides) can be employed for the diagnosis and therapy of neurological disorders. In addition, the diagnosis and treatment of genetic predispositions for particular neurological disorders such as epilepsy, ataxias, dystonia, stroke, psychological disorders such as anxiety, manic-depressive disorders, migraine, cognitive losses and other neurological disorders become possible. It is further possible to carry out a monitoring of the treatment of the abovementioned disorders.

A further aspect of the invention is a method for the qualitative and quantitative detection of a novel nucleic acid in a biological sample, which comprises the following steps:

a) incubation of a biolgical sample with a known amount of novel nucleic acid or a known amount of oligonucleotides which are suitable as primers for amplification of the novel nucleic acid,

b) detection of the novel nucleic acid by specific hybridization or PCR amplification,

c) comparison of the amount of hybridizing nucleic acid, or of nucleic acid obtained by PCR amplification, with a quantity standard.

The invention additionally relates to a method for the qualitative and quantitative detection of the novel protein complex or of a novel protein in a biological sample, which comprises the following steps:

a) incubation of a biological sample with an antibody which is specifically directed against the protein complex or against the novel protein,

b) detection of the antibody/antigen complex,

c) comparison of the amounts of the antibody/antigen complex with a quantity standard.

A biological sample from a healthy organism is normally taken as standard.

The invention further relates to a method for discovering substances which bind specifically to a protein having the amino acid sequence SEQ ID NO: 2 or SEQ ID NO: 4, which comprises one or more of the following steps:

a) expression of the protein in eukaryotic or prokaryotic cells,

b) incubation of the protein with the substances to be tested,

c) detection of the binding of a substance to Mogli or the domains in GIRK1 responsible for the intramolecular interaction, or of an effect on the K⁺ current or of a change in transmitter release.

The invention additionally relates to a method for discovering substances which specifically bind to a protein having an amino acid sequence shown in SEQ ID NO: 2 or SEQ ID NO: 4 or to a nucleic acid sequence shown in SEQ ID NO: 1 or SEQ ID NO: 3 and thus cause inhibiting or activating functional effects on the K⁺ conductivity in CNS neurons or on transmitter release.

In situations where there is a prevailing deficiency in the activity of the novel protein or of the GIRK1/Mogli complex completed with SEQ ID NO: 2 or SEQ ID NO: 4 it is possible to employ several methods for replacement. On the one hand, the protein, natural or recombinant, can be administered directly or, through suitable measures, in the form of its coding nucleic acid (i.e. DNA or RNA). It is possible to use for this purpose both viral and nonviral vehicles. A further way is provided by stimulation of the endogenous gene by suitable substances. Such substances can be discovered, for example, by measuring their effect on the transcription elements of the novel Mogli gene.

In situations where there is a prevailing excess of activity of the GIRK-protein complex comprising a protein having the sequence SEQ ID NO: 2 or SEQ ID NO: 4, or of a protein having SEQ ID NO: 2 or SEQ ID NO: 4 alone, it is possible to employ specific, synthetic or natural, competitive and non-competitive antagonists of the protein having the sequence SEQ ID NO: 2 or SEQ ID NO: 4 or antibodies or antibody fragments against the protein having the sequence SEQ ID NO: 2 or SEQ ID NO: 4 or against the protein complex. It is additionally possible to achieve an alteration in the GIRK currents or in the activity of the protein having the sequence SEQ ID NO: 2 or SEQ ID NO: 4 both through antisense molecules or ribozymes or oligonucleotides and through low molecular weight compounds.

Methods like those mentioned can be employed where there is a deficiency or excess of complexes between the novel protein and proteins involved in transmitter release.

In the novel method, the interacting protein is preferably a SNARE complex protein or a protein associated therewith or a Kir protein, and the method may also include the steps in the abovementioned methods.

Likewise included is a novel method where the substances enhance or diminish transmitter release. Transmitter release can be tested as described below.

The invention also relates in particular to drug products which comprise the novel nucleic acid sequence, the novel protein, the novel antibodies or the protein complexes, an antisense molecule of the novel nucleic acid sequence, or a substance which has been discovered by one of the preceding methods, and optionally a pharmaceutically suitable carrier.

The invention likewise relates to a method for detecting a disorder, which comprises the steps of the method, with the standard having been selected so that it represents the expression of a healthy organism.

Also included is a means for diagnosing genotypes which comprises said nucleic acid, a fragment thereof, or an antisense nucleic acid molecule thereof.

Accordingly, the invention also relates to a method for producing a drug product, which includes the steps of one of the novel methods and additionally comprises formulation of the discovered substance with a pharmaceutically suitable carrier. General methods are sufficiently well known to the skilled worker.

Finally, the invention relates to the use of a nucleic acid sequence, the protein, of the antibody or of an antisense molecule as have been described above, or of one of the substances discovered by the preceding methods, for producing a drug product for the treatment of neurological disorders. It can be used in particular to treat the abovementioned disorders based on increased or reduced transmitter release. Likewise, various disorders which can be influenced by modulation of GIRK proteins and which have been listed above can be treated.

### EXAMPLES

Molecular characterization of interaction partners of the GIRK1 subunit makes it possible to understand better the physiological and pharmacological diversity of the GIRK channels, and to obtain novel specific points of attack for pharmacotherapeutic interventions. GIRK1 interaction partners were found in a cDNA rat brain library by screening with the yeast two-hybrid system. Two overlapping fragments of an unknown cDNA were isolated. Using these fragments, two cDNAs about 3 kb in length were isolated by homology screening from a rat hippocampus and cortex cDNA library and then sequenced. The cDNA sequence SEQ ID NO: 1 obtained in this way contains the complete coding region for the sequence SEQ ID NO: 2.

Sequence analysis of the polypeptide encoded by the present cDNA (=SEQ ID NO: 1) predicts a large number of potential phosphorylation sites, a highly charged domain, and two hydrophobic domains in the carboxy-terminal region. It is possible that the latter extend through the plasma membrane, so that the probability is that it is a membrane protein. Several potential phosphorylation sites indicate that the protein itself is subject to regulation by phosphatases and/or kinases.

The charged domain, for which an α-helical structure is predicted, was identified in yeast cotransformation studies as the region interacting with GIRK1.

Sequence comparisons (Altschul et al., 1990) of the base sequence (SEQ ID NO: 1) with public nucleotide databases (embl) using the BLAST program (Version BLASTP 2.Oa19-WashU [05-Feb-1998]) showed similarities with a known cDNA sequence (“KIAA0374”; accession no: AB002372). At the amino acid level, the similarity is particularly great in the charged domain but does not extend over the amino-terminal region.

The proteins described in SEQ ID NO: 2 and SEQ ID NO: 4 are novel proteins which are able to interact with the GIRK1 subunit and have an influence on the properties of these channels.

The distribution of the mRNA from which the cDNA sequence SEQ ID NO: 1 originated has been investigated by Northern blot and by in situ hybridization on rat brain sections. Analysis of 10 different rat tissues revealed brain-specific expression of a mRNA 3 kb in size. The in situ hybridization revealed strong expression in the cortex, cerebellum and hippocampus, and weaker expression in other regions of the brain.

The expression pattern of SEQ ID NO: 1 overlaps with that of the GIRK1 subunit and indicates an important CNS function of the protein depicted in SEQ ID NO: 2. Unless stated otherwise, the experimental procedures corresponded to the methods in Ausubel et al., (eds.), 1998. Current Protocols in Molecular Biology. John Wiley & Sons, New York.

### Example 1

Two-hybrid search with the amino terminus and carboxyl terminus of the GIRK1 subunit

The cDNAs coding for the carboxyl terminus and amino terminus of the GIRK1 subunit (accession no.: U09243, EMBL database) were amplified from rat brain cDNA in two independent polymerase chain reactions (PCR) with the following specific primers:

GIRK1-NC-s
(5′-ACAGTCGACTATGTCTGCACTCCGAAGGAA-3′) and
GIRK1-NC-Las
(5′-ACCGCTGGAGCCCGAAGAGATAAAGAGGTTCCAAC-3′)

and, respectively,

GIRK1-NC-Ls
(5′-TCTTCGGGCTCCAGCGGTATCAAGATCTCCCAGCCC-3′) and
GIRK1-NC-as
(5′-GTCACTAGTGGTGTTTTGCTATGTGAAGCG-3′)

The resulting PCR products were fused in a further PCR by carrying out in a manner known to the skilled worker five reaction cycles with the enzyme/buffer mixture and the PCR products. In this reaction, the linker sequences attached to the primers GIRK1-NC-Las and GIRK1-NC-Ls served as primers for the complementary strand. The primers GIRK1-NC-s und GIRK1-NC-as were pipetted in for the following twenty reaction cycles. The resulting construct was digested with the restriction enzymes SalI and SpeI and then cloned via the protruding ends into a vector pDBLeu (supplied by LifeTechnologies) which had previously been cut with SalI and SpeI. The DNA construct produced in this way (“GIRK1-NC-bait”) codes for a protein in which the GAL4-DNA binding domain is fused to the N terminus of the GIRK1 subunit, a linker sequence (NH₂-GSSGSS-COOH) and to the C terminus of GIRK1. This construct was used to transform the yeast strain Y190 (supplied by LifeTechnologies). The resulting yeast strain was transformed with a rat brain cDNA bank in the vector pPC86 (supplied by LifeTechnologies), and 5.27 million transformants were plated out on tryptophan/leucine/histidine-deficient media provided with 20 mM 3-amino-1,2,4-triazole (3AT, supplied by SIGMA). After growth at 30° C. for 3, 4 and 5 days, eleven colonies with a diameter of 1 mm and more were isolated (GIRK1NC-preys 1-11) and subjected to X-Gal staining. A total of six colonies proved to be His3- and LacZ-positive. The bank plasmids were isolated from the latter, and the cDNA was sequenced using the vector-specific primers

| | |
|---|---|
| pPC86a | (5′-GTATAACGCGTTTGGAATCAC-3′) and |
| pPC86b | (5′-GTAAATTTCTGGCAAGGTAGAC-3′). |

Sequence analysis revealed 2 different overlapping fragments (“GIRK1NCprey3”: nucleotides 500-1193 and “GIRK1NCprey2”: nucleotides 1011-1349 of SEQ ID NO: 1) of an unknown cDNA.

The purified pPC86 plasmid DNA was cotransformed with various pDBLeu constructs into the yeast strain Y190. Only in combination with the construct GIRK1NC-bait was it possible to detect activation of the His3 and LacZ reporter genes.

### Example 2

Cloning of the cDNA for the novel GIRK1 interaction partner Mogli

A cDNA fragment obtained from the two-hybrid search as in Example 1 (nucleotides 500-1193 in sequence SEQ ID NO: 1) was radiolabeled with α-³²P-dCTP using the random primer labeling kit (supplied by BOEHRINGER MANNHEIM) in accordance with the manufacturer's instructions. The heat-denatured radioactive probe was hybridized (42° C., 5×SSC/50% formamide) for 16 hours on 20 nitrocellulose filters onto each of which 24000 plaques of a cDNA bank from rat hippocampus and cortex in bacteriophage λ had been transferred, and then washed several times with 0.2×SSC at 55° and 60° C. Nine of 20 positive λ clones were isolated, and phage DNA was isolated and mapped. The cDNA fragments of clones 8 and 11 were completely sequenced. They contain an open reading frame for amino acid sequence SEQ ID NO: 2. Sequence analysis of these two λ cDNA clones revealed a difference in the coding sequence, a silent mutation (nucleotide 1815: C to T). Analysis of the other phage clones revealed that the base T occurs in position 1815 in eight of nine investigated cases. SEQ ID NO: 1 thus includes sequences containing a T or a C at position 1815 (identified by “Y” in SEQ ID NO: 1). This is therefore a silent mutation which has no effect on the amino acid sequence of the protein. The amino acid concerned in both cases is proline (see Xaa in the sequences SEQ ID NO: 1 and SEQ ID NO: 2).

### Example 3

Expression of the mRNA for the novel GIRK1 interaction partner Mogli in rat tissues

A cDNA fragment obtained from the two-hybrid search as in Example 1 (nucleotides 500-1193 in sequence SEQ ID NO: 1) were radiolabeled with α-³²P-dCTP using the random primer labeling kit (supplied by BOEHRINGER MANNHEIM) in accordance with the manufacturer's instructions. The heat-denatured radioactive probe was incubated with a multiple tissue Northern blot (10 μg each of total RNA from rat brain, liver, lung, heart, kidney, skeletal muscle, small intestine and testis; isolated as described by Chomzinsky and Sacci, Anal. Biochem., 162, 156-159, 1987) in QuickHyb solution (supplied by STRATAGENE) at 68° C. for one hour and then washed with 0.1×SSC/0.1% SDS at 55° C. After exposure for 3 days, a strong hybridization signal was found for brain RNA at about 3 kb and no signal in any of the other tissues investigated (see FIG. 1, A). FIG. 1A: Depiction of the multiple tissue Northern produced as described above. The migration distances of the RNA size standard used (in kilobases), and the organs of origin of the RNA samples loaded in the various lanes are indicated.

The same probe was incubated with a Northern blot of various stages of development (10 μg each of total RNA from rat brain in the stages embryonic (E) 9, E12, E15, E18, postnatal (P) 0, P14, P21 and adult; isolated by the method of Chomzinsky and Sacci (see above) in QuickHyb solution (supplied by STRATAGENE) at 68° C. for one hour and then washed with 0.1×SSC/0.1% SDS at 55° C. After exposure for 3 days, no hybridization signal was detected in stage E9; a weak hybridization signal was detected in stage E12 and a distinct signal from E15 onwards, which increases slightly up to the adult stage (see FIG. 1, B). FIG. 1B: Depiction of the Northern blot at various stages of development which was produced as described above. The migration distances of the RNA size standard used (in kilobases) and the age of the rats whose brain RNA samples were loaded in the various lanes are indicated.

### Example 4

Expression of the mRNA for the novel GIRK1 interaction partner Mogli in rat brain

Distribution of the mRNA for SEQ ID NO: 1 in the rat brain was established by in situ hybridization using RNA probes derived from SEQ ID NO: 1. Sense and antisense probes were produced with suitable RNA polymerases T7 and T3 and digoxigenin-labeled nucleotides (UTP) from a pBS vector which contained SEQ ID NO: 1 and had been linearized with XhoI or NotI. About 15 μm thick horizontal brain sections were fixed, permeabilized, acetylated and hybridized with the probes in 5×SSC, 50% formamide at 65° C. overnight. The sections were washed twice for 10 minutes with 2×SSC at 20° C., then for 10 minutes with 0.2×SSC at 65° and finally for 5 minutes with 0.2×SSC at 20° C. Single-stranded RNA was then degraded by treatment with RNAseA (50 μg/ml) (supplied by SIGMA) at 37° C. for 30 minutes. In accordance with the manufacturer's instructions, the sections were incubated with alkaline phosphatase-labeled anti-digoxigenin antibodies (BOEHRINGER MANNHEIM), washed and detected.

The strongest signal was detected in Purkinje cells and granule cells of the cerebellum. Strong signals were likewise found in the cortex and in the hippocampus, and weaker ones were found in various thalamic nuclei, in the striatum and in the midbrain (see FIG. 2). FIG. 2: In situ hybridization for SEQ ID NO: 1, FIG. 2A: Horizontal section through adult rat brain. SEQ ID NO: 1 hybridization signals appear light because of the inverse representation.

FIG. 2 B-D: Enlarged details of a horizontal section through an adult rat brain. Comparison of the hybridization patterns of SEQ ID NO: 1 and GIRK1 in the cerebellum and hippocampus. Hybridization signals are shown dark in this case (abbreviations in the figure: CA1-3: hippocampus fields CA1-3; Ctx: cortex; Dg: dentate gyrus; Gr: granular layer/granule cells; Hi: hippocampus; Pu: Purkinje cells).

### Example 5

Cloning and sequencing of the cDNA for the human form of the novel GIRK1 interaction partner Mogli (“hsMogli”)

A cDNA fragment obtained from the two-hybrid search as in Example 1 (nucleotides 500-1193 in sequence SEQ ID NO: 1) was radiolabeled with α-³²P-dCTP using the random primer labeling kit (supplied by BOEHRINGER MANNHEIM) in accordance with the manufacturer's instructions. The heat-denatured radioactive probe was hybridized (42° C., 5×SSC/50% formamide) for 16 hours on 18 nitrocellulose filters onto each of which 30000 plaques of a cDNA bank from human hippocampus in bacteriophage λ had been transferred, and then washed with 1×SSC at 20° for 15 minutes and twice with 0.5×SSC at 50° C. for 10 minutes each time. Four positive λ clones were isolated, and phage DNA was isolated and mapped. The cDNA fragments of the two longest clones (1 and 2) were completely sequenced. They contained the hsMogli sequence corresponding to nucleotides 751-1992 and 707-1992, respectively, in SEQ ID NO: 3. None of the isolated clones contained sequences located further upstream. In order to complete the open reading frame for hsMogli, human hippocampal RNA was transcribed into cDNA with reverse transcriptase (supplied by LIFETECHNOLOGIES). Starting from this cDNA, various PCR reactions were carried out with KlenTaq DNA polymerase (supplied by CLONTECH) in accordance with the manufacturer's instructions using primer oligonucleotides derived from the rat sequence SEQ ID NO: 1 and from public human database sequences (ESTs). One of these reactions, hsMogli8s(5′-TCGCACAGCTGATAGGATTAGG-3′)/hsMoglillas(5′- CCTCATGATGGGGCTACAGTCG -3′),

resulted in a truncated product whose sequence did, however, make it possible to synthesize specific primers for hsMogli. The primer combinations

| | |
|---|---|
| hsMogli12s | (5′-TTTGTCAGCCCTGATTGAGCC-3′)/ |
| hsMogli14as | (5′-GAGCTGCTGGGGTTGAACTCTC-3′), |
| hsMogli13s | (5′-GAGAGTTCAACCCCAGCAGCTC-3′)/ |
| hsMogli11as | (5′-CCTCATGATGGGGCTACAGTCG-3′) and |
| hsMogli23s | (5′-GAGAGCAAGGAGCACAGA-3′)/ |
| hsMogli11as | (5′-CCTCATGATGGGGCTACAGTCG-3′) |

finally produced the required band sizes. The amplicons were directly sequenced with the PCR primers and with internal primers. The resulting sequence was fused to the sequence from the λ clones 1 and 2. It produced an open reading frame which is depicted in SEQ ID NO:3.

### Example 6

Cotransformation studies for more accurate determination of the interacting domains of GIRK1 and the GIRK1 interaction partner Mogli

The DNA (accession no.: U09243, EMBL datebase) coding for the carboxyl terminus or the amino terminus of the GIRK1 subunit was amplified by PCR starting from the plasmid “GIRK1NC-bait” using the primers GGIRK1-4s (5′-TAGGTCGACCATGTTTAGCGAGCATGCGGTT-3′) and GGIRK1-as (5′-GTCACTAGTTGGGGTGTTTTGCTATGTGAAG-3′) or GIRK1-NC-s (5′ -ACAGTCGACTATGTCTGCACTCCGAAGGAA-3′) and GIRK1-N-as (5′-GTCACTAGTGATAAAGAGGTTCCAAC-3′) and cloned into the vector pDBLeu. Each of the plasmids was cotransformed with the pPC86 plasmid. GIRK1NCprey2 (SEQ ID NO: 1 nucleotides 1011-1349) or with the plasmid GIRK1NCprey3 (SEQ ID NO: 1, nucleotides 500-1193) into the yeast strain Y190. Cotransformation of the N or C terminus of GIRK1 with one of the prey plasmids did not in any case lead to activation of the reporter genes of the yeast strain Y190. It is evident that the interaction with Mogli takes place only in the presence of both intracellular portions of GIRK1.

The nucleotide region 1011-1193 of SEQ ID NO: 1 is present in both of the plasmids GIRK1NCprey2 and GIRK1NCprey3. In order to check whether this region is able to mediate an interaction with GIRK1, it was cloned into the plasmid pPC86 using the primers 1NCprey23o-s (5′-ACAGTCGACGGAGTCTGAGCGCCGA-3′) and 1NCprey23o-as (5′-GTCGCGGCCGCCTGCTCCTCATAGTCTC-3′).

This construct was cotransformed with the clone GIRK1-NC-bait into the yeast strain Y190, and activation of the reporter genes was checked. Once again, no activation took place, which indicates that a larger element of the Mogli protein must be responsible for a functional interaction.

The public databases contain a human sequence (“KIAA0374”; accession no.: AB002372) which codes for a protein which shows in the region of amino acids 85-243 similarity with amino acids 237-395 in SEQ ID NO: 2 (comprising SEQ ID NO: 6) and with amino acids 233-391 in SEQ ID NO: 4 (comprising SEQ ID NO: 8). These regions in the three proteins are distinguished by a large number of charged amino acids, and structure prediction programs indicated an α-helical structure for this region. The DNA for the charged domains of SEQ ID NO: 6 and amino acid 85-243 in KIAA0374 were amplified using specific primers, digested with the restriction enzymes SalI/NotI and cloned into a pPC86 vector digested in the same way (MOGLI-charged-s: 5′-GACGTCGACAGGCTCCTACAAAGGAAGCGAC-3′ and MOGLI-charged-as: 5′-GAGCGGCCGCTCAGTCTAGACACAGTTCATCCCTC-3′; MOGLI2-charged-s: 5′-GACGTCGACAGGCTCCTACAAGGGCAGTGAC-3′ and MOGLI2-charged-as: 5′ -GAGCGGCCGCTCACTCCCCAGTGCCATCCTCCTT-3′). These constructs were cotransformed with GIRK1-NC-bait into the yeast strain Y190. Activation of the His3 and LacZ reporter genes was detectable only in the combination of GIRK1NC-bait with the construct of the charged part of Mogli. The charged domain of the Mogli protein was accordingly able to interact with GIRK1; the result underlines the specificity of the GIRK1I-gli interaction, because no interaction with GIRK1 was detectable for the charged domain of the protein encoded by KIAA0374, despite great

### Example 7

Two-hybrid search using the amino terminus of SEQ ID NO: 1

The cDNA coding for the amino terminus of SEQ ID NO: 1 was amplified in a polymerase chain reaction (PCR) with the specific primers GIP-N-s (5′CGGAATTCGCAGGCAACGACGAGATG-3′) and GIP-N-as (5′-CGCGTCGACGTCTAGACACATGTCATCC-3′) from the cDNA fragment of λ8 (Example 2). The resulting construct was digested with the estriction enzymes SalI and EcoRI and then cloned via the rotruding ends into a pGBT10 vector (supplied by CLONTECH) which had previously been cut with SalI and EcoRI. The DNA construct produced in this way (“MogliN-bait”) codes for a protein in which the GAL4 DNA-binding domain is fused to the N terminus of SEQ ID NO: 2. This construct was used to transform the yeast strain HF7c (supplied by CLONTECH). The resulting yeast strain was transformed with a rat brain cDNA bank in the vector pACT2 (supplied by CLONTECH), and 1.25 million transformants were plated out onto tryptophan/leucine/histidine-deficient media. After growth for 3, 4 and 5 days at 21° C., colonies with a diameter of 1 mm and more were isolated (MogliN-preys 1-210) and subjected to staining with X-Gal. A total of 54 colonies proved to be His3- and LacZ-positive. The bank plasmids were isolated therefrom, and the cDNA was sequenced using the vector-specific primers pACT2s (5′-CTATTCGATGATGAAGATACCCCACCAAACCC-3′) and pACT2as (5′-GTGAACTTGCGGGGTTTTTCAGTATCTACGA-3′). The sequence analysis revealed the following:

a) 11 independent clones contained the cDNA sequence which codes for the rat homolog of the database entries for Mus musculus EB2 (accession No: U51204) and Homo sapiens EB3 (accession No: AB025186)

b) 3 independent clones contained the cDNA sequence which codes for the rat homolog of the database entry for Homo sapiens KIAA0627 (accession No: AB014527)

c) 2 independent clones contained the cDNA sequence which codes for the rat homolog of the database entry for Homo sapiens KIAA0622 (accession No: AB014522).

The purified pACT2 plasmid DNAs were cotransformed with various pGBT10 constructs into the yeast strain HF7c. Activation of the reporter genes His3 and LacZ was detectable only in combination with the construct MogliN-bait.

```
′
# SEQUENCE LIS
#TING
<160> NUMBER OF SEQ ID NOS: 34
<210> SEQ ID NO 1
<211> LENGTH: 2841
<212> TYPE: DNA
<213> ORGANISM: Rattus norvegicus
<220> FEATURE:
<221> NAME/KEY: CDS
<222> LOCATION: (142)..(2139)
<400> SEQUENCE: 1
cttgctcgca cagctgatag gattaggagc ccgtgctttg tcggccctga tt
#gagtccaa 60
gacagccccg gcatacggca tacacaggtg cctcctcctg gacggcggcg gc
#ggcgcggg 120
gagcctgcag gcaacgacga g atg gga ccc ctc cga gag
#agc aag aag gag 171
# Met Gly Pro Leu Arg Glu Ser
#Lys Lys Glu
# 1
# 5
# 10
cag aga gtc cag cat cag gag aag gag ttc tc
#c agg agc cgg att ccc 219
Gln Arg Val Gln His Gln Glu Lys Glu Phe Se
#r Arg Ser Arg Ile Pro
                                  15
# 20
# 25
agg ttg att ctg cga ccc cat ctg cct cag ca
#g cag cag cag cag cag 267
Arg Leu Ile Leu Arg Pro His Leu Pro Gln Gl
#n Gln Gln Gln Gln Gln
                          30
# 35
# 40
aac aag gtt tcc cca gcc tcc gag tct ccc tt
#t tca gag gaa gaa agt 315
Asn Lys Val Ser Pro Ala Ser Glu Ser Pro Ph
#e Ser Glu Glu Glu Ser
                  45
# 50
# 55
aga gag ttc aac ccc agc agc tcc gga cgt tc
#a gca agg aca att agc 363
Arg Glu Phe Asn Pro Ser Ser Ser Gly Arg Se
#r Ala Arg Thr Ile Ser
          60
# 65
# 70
agc aac agc ttc tgc tca gac gac aca ggt tg
#t ccc agc agc cag tcg 411
Ser Asn Ser Phe Cys Ser Asp Asp Thr Gly Cy
#s Pro Ser Ser Gln Ser
  75
# 80
# 85
# 90
gta tcc cct gtg aag act ccc tca gac act gg
#a cac agt ccc att ggc 459
Val Ser Pro Val Lys Thr Pro Ser Asp Thr Gl
#y His Ser Pro Ile Gly
                                  95
# 100
# 105
ttt tgc cct gga agt gat gaa gat ttt acc ag
#g aag aaa tgc agg att 507
Phe Cys Pro Gly Ser Asp Glu Asp Phe Thr Ar
#g Lys Lys Cys Arg Ile
                        110
# 115
# 120
ggg atg gtt ggt gag gga aat atc caa tca gc
#t cgt tat aaa aaa gaa 555
Gly Met Val Gly Glu Gly Asn Ile Gln Ser Al
#a Arg Tyr Lys Lys Glu
                125
# 130
# 135
tcc aag gga ggc atc ata aag cca ggt agt ga
#a gca gat ttt agc tcc 603
Ser Lys Gly Gly Ile Ile Lys Pro Gly Ser Gl
#u Ala Asp Phe Ser Ser
        140
# 145
# 150
tca agc agc aca ggc agc atc tcg gct cct ga
#g gtc cac atg tcc acg 651
Ser Ser Ser Thr Gly Ser Ile Ser Ala Pro Gl
#u Val His Met Ser Thr
155 1
#60 1
#65 1
#70
aca gga aac aag cga gcc tct ttc tca cgc aa
#c aga ggt cct cat ggg 699
Thr Gly Asn Lys Arg Ala Ser Phe Ser Arg As
#n Arg Gly Pro His Gly
                                175
# 180
# 185
cgg agc aat gga gca cca tcc cac aag tct gg
#c agc agc cca ccg tcc 747
Arg Ser Asn Gly Ala Pro Ser His Lys Ser Gl
#y Ser Ser Pro Pro Ser
                        190
# 195
# 200
cca agg gaa aaa gac ctt gtg tct atg ctg tg
#c aga aat cca ctg agc 795
Pro Arg Glu Lys Asp Leu Val Ser Met Leu Cy
#s Arg Asn Pro Leu Ser
                205
# 210
# 215
ccc agt aac atc cat cct agc tac gcc cct tc
#t tct cca agt agc agc 843
Pro Ser Asn Ile His Pro Ser Tyr Ala Pro Se
#r Ser Pro Ser Ser Ser
        220
# 225
# 230
aac tcc ggc tcc tac aaa gga agc gac tgt ag
#t cca gtc atg agg agg 891
Asn Ser Gly Ser Tyr Lys Gly Ser Asp Cys Se
#r Pro Val Met Arg Arg
235 2
#40 2
#45 2
#50
tct gga cga tat atg tct tgt gga gaa aat ca
#t ggc gtc aaa ccc cca 939
Ser Gly Arg Tyr Met Ser Cys Gly Glu Asn Hi
#s Gly Val Lys Pro Pro
                                255
# 260
# 265
aat cca gaa cag tat ttg aca cct ctg cag ca
#g aag gag gtc aca gtg 987
Asn Pro Glu Gln Tyr Leu Thr Pro Leu Gln Gl
#n Lys Glu Val Thr Val
                        270
# 275
# 280
agg cat ttg agg acc aag ctg aag gag tct ga
#g cgc cga ctc cat gag 1035
Arg His Leu Arg Thr Lys Leu Lys Glu Ser Gl
#u Arg Arg Leu His Glu
                285
# 290
# 295
agg gaa tct gaa atc atg gag ctc aag tct ca
#g ctg gct cgg atg agg 1083
Arg Glu Ser Glu Ile Met Glu Leu Lys Ser Gl
#n Leu Ala Arg Met Arg
        300
# 305
# 310
gaa gac tgg ata gag gaa gag tgc cac agg gt
#g gag gct cag ttg gcg 1131
Glu Asp Trp Ile Glu Glu Glu Cys His Arg Va
#l Glu Ala Gln Leu Ala
315 3
#20 3
#25 3
#30
ctc aaa gaa gcc aga aaa gag att aag cag ct
#c aaa cag gtc att gag 1179
Leu Lys Glu Ala Arg Lys Glu Ile Lys Gln Le
#u Lys Gln Val Ile Glu
                                335
# 340
# 345
act atg agg agc agc ttg gct gat aaa gat aa
#a ggc att cag aag tac 1227
Thr Met Arg Ser Ser Leu Ala Asp Lys Asp Ly
#s Gly Ile Gln Lys Tyr
                        350
# 355
# 360
ttt gtg gac ata aac atc caa aac aag aaa ct
#g gag tct ctg ctt caa 1275
Phe Val Asp Ile Asn Ile Gln Asn Lys Lys Le
#u Glu Ser Leu Leu Gln
                365
# 370
# 375
agc atg gag atg gcg cac aat agt tcc ctg ag
#g gat gaa ctg tgt cta 1323
Ser Met Glu Met Ala His Asn Ser Ser Leu Ar
#g Asp Glu Leu Cys Leu
        380
# 385
# 390
gac ttc tcc ttc gat tcc cca gag aaa agc tt
#a ccc cta agc agc aca 1371
Asp Phe Ser Phe Asp Ser Pro Glu Lys Ser Le
#u Pro Leu Ser Ser Thr
395 4
#00 4
#05 4
#10
tat gac aag atg gcg gac ggg ttg tct ctg ga
#a gaa cag ata aca gag 1419
Tyr Asp Lys Met Ala Asp Gly Leu Ser Leu Gl
#u Glu Gln Ile Thr Glu
                                415
# 420
# 425
gaa ggt gct gac agt gag ctt ctg gtg gga ga
#c agc atg gcc gag ggc 1467
Glu Gly Ala Asp Ser Glu Leu Leu Val Gly As
#p Ser Met Ala Glu Gly
                        430
# 435
# 440
aca gat ctg tta gat gag ata gtg act gcc ac
#c acc aca gaa tcc ggt 1515
Thr Asp Leu Leu Asp Glu Ile Val Thr Ala Th
#r Thr Thr Glu Ser Gly
                445
# 450
# 455
gac ctg gag ttt gtt cat tcc act cca ggg cc
#a caa gcc ctc aag cct 1563
Asp Leu Glu Phe Val His Ser Thr Pro Gly Pr
#o Gln Ala Leu Lys Pro
        460
# 465
# 470
ctc ccc ttg gtg agc cag gaa gag ggc att gt
#g gtg gtg gag caa gca 1611
Leu Pro Leu Val Ser Gln Glu Glu Gly Ile Va
#l Val Val Glu Gln Ala
475 4
#80 4
#85 4
#90
gtg cag acc gat gtg gtg ccg ttc agc cct gc
#c atc tca gag ctc ctt 1659
Val Gln Thr Asp Val Val Pro Phe Ser Pro Al
#a Ile Ser Glu Leu Leu
                                495
# 500
# 505
cag agt gtg cta aag ttg cag gac tcc tgt cc
#c aca agc tca gca tcc 1707
Gln Ser Val Leu Lys Leu Gln Asp Ser Cys Pr
#o Thr Ser Ser Ala Ser
                        510
# 515
# 520
cca gat gaa tcc aga gct gac tca atg gaa ag
#c ttc tca gaa tcc atc 1755
Pro Asp Glu Ser Arg Ala Asp Ser Met Glu Se
#r Phe Ser Glu Ser Ile
                525
# 530
# 535
tct gcc tta atg gtt gat tta act cca aga ag
#t ccc aac tca gcc atc 1803
Ser Ala Leu Met Val Asp Leu Thr Pro Arg Se
#r Pro Asn Ser Ala Ile
        540
# 545
# 550
ctt ctg tct ccy gtg gag att cca ttc agc aa
#g gca gct acg gaa gcc 1851
Leu Leu Ser Arg Val Glu Ile Pro Phe Ser Ly
#s Ala Ala Thr Glu Ala
555 5
#60 5
#65 5
#70
cgt gca aac cgc ctc atg aga gag cta gat tt
#t gca gcc tgc aca gaa 1899
Arg Ala Asn Arg Leu Met Arg Glu Leu Asp Ph
#e Ala Ala Cys Thr Glu
                                575
# 580
# 585
gaa agg ttg gac agc atc ctc tcg ctg tct ca
#g gga ggt gtc gtg agg 1947
Glu Arg Leu Asp Ser Ile Leu Ser Leu Ser Gl
#n Gly Gly Val Val Arg
                        590
# 595
# 600
cag tac tgg agc agc agt ttc ttg gtg gat ct
#a ctg gct gtg gct gcc 1995
Gln Tyr Trp Ser Ser Ser Phe Leu Val Asp Le
#u Leu Ala Val Ala Ala
                605
# 610
# 615
cct gtg gta ccc act gtt ttg tgg gta ttc ag
#t act cag aga ggg ggt 2043
Pro Val Val Pro Thr Val Leu Trp Val Phe Se
#r Thr Gln Arg Gly Gly
        620
# 625
# 630
aca gat cct gtc tac aac att gga gcc ctg ct
#c cgg ggc tgc tgt gtg 2091
Thr Asp Pro Val Tyr Asn Ile Gly Ala Leu Le
#u Arg Gly Cys Cys Val
635 6
#40 6
#45 6
#50
gtg gct cta cac tcc cta cgc cgc aca gct tt
#c cac atg aaa acc taa 2139
Val Ala Leu His Ser Leu Arg Arg Thr Ala Ph
#e His Met Lys Thr
                                655
# 660
# 665
ttagtcgtta ccatgtgcca atgtatctgt gtagcgtggt gccaggtaga gc
#aacctcag 2199
gtggatcagt ggaagtctct attgtcattt ttgctccttg ctatttgatt tg
#cactatag 2259
tcagttgcag cctgttcact gtttaaacca gaggtatctt ccaaggcatg ga
#aacctggt 2319
tctggtagat gtcccaccag agtggcgtag aaagcatgct tgtgcccctg cc
#gtgttgtc 2379
tgaggtgccc gttcttatac taatggttca gaaagagaaa atgcagtttg ca
#ctttcacc 2439
acagcctctc taaggctggg catgttatct ccttgctttg ctttgtgctg tt
#ttaaaatg 2499
tgtaattgtt ccagcattcc aatggtcttg tgcatagcag gggactgtaa cc
#aaaaataa 2559
aaatgtattt gtgtaattag ttcaaagaag actcgaatag ctctttattg tc
#tttcttgg 2619
ggttgataaa gtttgagtgt ttggattttt ttttaaatgt agctccaaag tc
#ttaaaagg 2679
ctcatttgct cttaaacctg tcagttgatg atactatgta aatttacaat gt
#actaactt 2739
attttttgct tattatatat agtggttctt tttttggaaa ttatttgtac cc
#acacactt 2799
cagcatgaaa ataaagatta gtgtttccat ttaaaaaaaa aa
#
#2841
<210> SEQ ID NO 2
<211> LENGTH: 665
<212> TYPE: PRT
<213> ORGANISM: Rattus norvegicus
<400> SEQUENCE: 2
Met Gly Pro Leu Arg Glu Ser Lys Lys Glu Gl
#n Arg Val Gln His Gln
    1 5
# 10
# 15
Glu Lys Glu Phe Ser Arg Ser Arg Ile Pro Ar
#g Leu Ile Leu Arg Pro
                          20
# 25
# 30
His Leu Pro Gln Gln Gln Gln Gln Gln Gln As
#n Lys Val Ser Pro Ala
                  35
# 40
# 45
Ser Glu Ser Pro Phe Ser Glu Glu Glu Ser Ar
#g Glu Phe Asn Pro Ser
          50
# 55
# 60
Ser Ser Gly Arg Ser Ala Arg Thr Ile Ser Se
#r Asn Ser Phe Cys Ser
  65
# 70
# 75
# 80
Asp Asp Thr Gly Cys Pro Ser Ser Gln Ser Va
#l Ser Pro Val Lys Thr
                                  85
# 90
# 95
Pro Ser Asp Thr Gly His Ser Pro Ile Gly Ph
#e Cys Pro Gly Ser Asp
                        100
# 105
# 110
Glu Asp Phe Thr Arg Lys Lys Cys Arg Ile Gl
#y Met Val Gly Glu Gly
                115
# 120
# 125
Asn Ile Gln Ser Ala Arg Tyr Lys Lys Glu Se
#r Lys Gly Gly Ile Ile
        130
# 135
# 140
Lys Pro Gly Ser Glu Ala Asp Phe Ser Ser Se
#r Ser Ser Thr Gly Ser
145 1
#50 1
#55 1
#60
Ile Ser Ala Pro Glu Val His Met Ser Thr Th
#r Gly Asn Lys Arg Ala
                                165
# 170
# 175
Ser Phe Ser Arg Asn Arg Gly Pro His Gly Ar
#g Ser Asn Gly Ala Pro
                        180
# 185
# 190
Ser His Lys Ser Gly Ser Ser Pro Pro Ser Pr
#o Arg Glu Lys Asp Leu
                195
# 200
# 205
Val Ser Met Leu Cys Arg Asn Pro Leu Ser Pr
#o Ser Asn Ile His Pro
        210
# 215
# 220
Ser Tyr Ala Pro Ser Ser Pro Ser Ser Ser As
#n Ser Gly Ser Tyr Lys
225 2
#30 2
#35 2
#40
Gly Ser Asp Cys Ser Pro Val Met Arg Arg Se
#r Gly Arg Tyr Met Ser
                                245
# 250
# 255
Cys Gly Glu Asn His Gly Val Lys Pro Pro As
#n Pro Glu Gln Tyr Leu
                        260
# 265
# 270
Thr Pro Leu Gln Gln Lys Glu Val Thr Val Ar
#g His Leu Arg Thr Lys
                275
# 280
# 285
Leu Lys Glu Ser Glu Arg Arg Leu His Glu Ar
#g Glu Ser Glu Ile Met
        290
# 295
# 300
Glu Leu Lys Ser Gln Leu Ala Arg Met Arg Gl
#u Asp Trp Ile Glu Glu
305 3
#10 3
#15 3
#20
Glu Cys His Arg Val Glu Ala Gln Leu Ala Le
#u Lys Glu Ala Arg Lys
                                325
# 330
# 335
Glu Ile Lys Gln Leu Lys Gln Val Ile Glu Th
#r Met Arg Ser Ser Leu
                        340
# 345
# 350
Ala Asp Lys Asp Lys Gly Ile Gln Lys Tyr Ph
#e Val Asp Ile Asn Ile
                355
# 360
# 365
Gln Asn Lys Lys Leu Glu Ser Leu Leu Gln Se
#r Met Glu Met Ala His
        370
# 375
# 380
Asn Ser Ser Leu Arg Asp Glu Leu Cys Leu As
#p Phe Ser Phe Asp Ser
385 3
#90 3
#95 4
#00
Pro Glu Lys Ser Leu Pro Leu Ser Ser Thr Ty
#r Asp Lys Met Ala Asp
                                405
# 410
# 415
Gly Leu Ser Leu Glu Glu Gln Ile Thr Glu Gl
#u Gly Ala Asp Ser Glu
                        420
# 425
# 430
Leu Leu Val Gly Asp Ser Met Ala Glu Gly Th
#r Asp Leu Leu Asp Glu
                435
# 440
# 445
Ile Val Thr Ala Thr Thr Thr Glu Ser Gly As
#p Leu Glu Phe Val His
        450
# 455
# 460
Ser Thr Pro Gly Pro Gln Ala Leu Lys Pro Le
#u Pro Leu Val Ser Gln
465 4
#70 4
#75 4
#80
Glu Glu Gly Ile Val Val Val Glu Gln Ala Va
#l Gln Thr Asp Val Val
                                485
# 490
# 495
Pro Phe Ser Pro Ala Ile Ser Glu Leu Leu Gl
#n Ser Val Leu Lys Leu
                        500
# 505
# 510
Gln Asp Ser Cys Pro Thr Ser Ser Ala Ser Pr
#o Asp Glu Ser Arg Ala
                515
# 520
# 525
Asp Ser Met Glu Ser Phe Ser Glu Ser Ile Se
#r Ala Leu Met Val Asp
        530
# 535
# 540
Leu Thr Pro Arg Ser Pro Asn Ser Ala Ile Le
#u Leu Ser Arg Val Glu
545 5
#50 5
#55 5
#60
Ile Pro Phe Ser Lys Ala Ala Thr Glu Ala Ar
#g Ala Asn Arg Leu Met
                                565
# 570
# 575
Arg Glu Leu Asp Phe Ala Ala Cys Thr Glu Gl
#u Arg Leu Asp Ser Ile
                        580
# 585
# 590
Leu Ser Leu Ser Gln Gly Gly Val Val Arg Gl
#n Tyr Trp Ser Ser Ser
                595
# 600
# 605
Phe Leu Val Asp Leu Leu Ala Val Ala Ala Pr
#o Val Val Pro Thr Val
        610
# 615
# 620
Leu Trp Val Phe Ser Thr Gln Arg Gly Gly Th
#r Asp Pro Val Tyr Asn
625 6
#30 6
#35 6
#40
Ile Gly Ala Leu Leu Arg Gly Cys Cys Val Va
#l Ala Leu His Ser Leu
                                645
# 650
# 655
Arg Arg Thr Ala Phe His Met Lys Thr
                        660
# 665
<210> SEQ ID NO 3
<211> LENGTH: 1992
<212> TYPE: DNA
<213> ORGANISM: Homo sapiens
<220> FEATURE:
<221> NAME/KEY: CDS
<222> LOCATION: (1)..(1992)
<400> SEQUENCE: 3
atg ggg ccc ctc cgc gag agc aag aag gag ca
#c aga gtg cag cat cat 48
Met Gly Pro Leu Arg Glu Ser Lys Lys Glu Hi
#s Arg Val Gln His His
    1 5
# 10
# 15
gac aag gag att tct cga agc cga att ccc cg
#g ttg att ctt cgg ccc 96
Asp Lys Glu Ile Ser Arg Ser Arg Ile Pro Ar
#g Leu Ile Leu Arg Pro
                          20
# 25
# 30
cat atg ccc caa caa cag cac aaa gtg tcc cc
#a gcc tct gag tct cct 144
His Met Pro Gln Gln Gln His Lys Val Ser Pr
#o Ala Ser Glu Ser Pro
                  35
# 40
# 45
ttc tct gag gaa gag agc aga gag ttc aac cc
#c agc agc tct ggg cgc 192
Phe Ser Glu Glu Glu Ser Arg Glu Phe Asn Pr
#o Ser Ser Ser Gly Arg
          50
# 55
# 60
tca gcg agg acc gtt agc agc aac agc ttc tg
#c tca gat gac aca ggc 240
Ser Ala Arg Thr Val Ser Ser Asn Ser Phe Cy
#s Ser Asp Asp Thr Gly
  65
# 70
# 75
# 80
tgt cct agc agc cag tca gtg tct cct gtg aa
#g aca ccc tca gat gct 288
Cys Pro Ser Ser Gln Ser Val Ser Pro Val Ly
#s Thr Pro Ser Asp Ala
                                  85
# 90
# 95
gga aac agc ccc att ggc ttt tgc cct gga ag
#t gat gaa ggc ttc acc 336
Gly Asn Ser Pro Ile Gly Phe Cys Pro Gly Se
#r Asp Glu Gly Phe Thr
                        100
# 105
# 110
aga aag aaa tgc acg att gga atg gtt ggt ga
#a gga agc att cag tcc 384
Arg Lys Lys Cys Thr Ile Gly Met Val Gly Gl
#u Gly Ser Ile Gln Ser
                115
# 120
# 125
tct cga tat aag aag gaa tca aag tca ggc ct
#t gtg aaa cca ggt agt 432
Ser Arg Tyr Lys Lys Glu Ser Lys Ser Gly Le
#u Val Lys Pro Gly Ser
        130
# 135
# 140
gaa gct gat ttt agc tcc tcg agc agc aca gg
#c agc att tcc gct cct 480
Glu Ala Asp Phe Ser Ser Ser Ser Ser Thr Gl
#y Ser Ile Ser Ala Pro
145 1
#50 1
#55 1
#60
gag gtc cat atg tcg act gcg gga agc aag cg
#g tct tct tct tca cgc 528
Glu Val His Met Ser Thr Ala Gly Ser Lys Ar
#g Ser Ser Ser Ser Arg
                                165
# 170
# 175
aat cga ggt cct cat ggg cgg agt aat gga gc
#t tcg tca cac aag cct 576
Asn Arg Gly Pro His Gly Arg Ser Asn Gly Al
#a Ser Ser His Lys Pro
                        180
# 185
# 190
ggc agc agc cca tca tcc ccg cgg gaa aag ga
#c ctt ctg tcc atg ctg 624
Gly Ser Ser Pro Ser Ser Pro Arg Glu Lys As
#p Leu Leu Ser Met Leu
                195
# 200
# 205
tgc agg aat cag ctg agc cct gtc aat atc ca
#t ccc agt tat gca cct 672
Cys Arg Asn Gln Leu Ser Pro Val Asn Ile Hi
#s Pro Ser Tyr Ala Pro
        210
# 215
# 220
tct tcc cca agc agt agc aac tca ggc tcc ta
#c aaa gga agc gac tgt 720
Ser Ser Pro Ser Ser Ser Asn Ser Gly Ser Ty
#r Lys Gly Ser Asp Cys
225 2
#30 2
#35 2
#40
agc ccc atc atg agg cgt tct gga agg tac at
#g tct tgc ggt gaa aat 768
Ser Pro Ile Met Arg Arg Ser Gly Arg Tyr Me
#t Ser Cys Gly Glu Asn
                                245
# 250
# 255
cat ggt gtc aga ccc cca aac cca gag cag ta
#t ttg act cca ctg cag 816
His Gly Val Arg Pro Pro Asn Pro Glu Gln Ty
#r Leu Thr Pro Leu Gln
                        260
# 265
# 270
cag aaa gag gtg aca gtg aga cac ctc aaa ac
#c aag ctg aag gaa tct 864
Gln Lys Glu Val Thr Val Arg His Leu Lys Th
#r Lys Leu Lys Glu Ser
                275
# 280
# 285
gag cgc cga ctc cat gaa agg gaa agt gaa at
#c gtg gag ctt aag tcc 912
Glu Arg Arg Leu His Glu Arg Glu Ser Glu Il
#e Val Glu Leu Lys Ser
        290
# 295
# 300
cag ctg gcc cgc atg cga gag gac tgg att ga
#g gag gag tgt cac cgg 960
Gln Leu Ala Arg Met Arg Glu Asp Trp Ile Gl
#u Glu Glu Cys His Arg
305 3
#10 3
#15 3
#20
gta gag gcc cag ttg gca ctc aaa gaa gcc ag
#g aaa gag att aaa cag 1008
Val Glu Ala Gln Leu Ala Leu Lys Glu Ala Ar
#g Lys Glu Ile Lys Gln
                                325
# 330
# 335
ctc aaa cag gtc atc gaa acc atg cgg agc ag
#c ttg gct gat aaa gat 1056
Leu Lys Gln Val Ile Glu Thr Met Arg Ser Se
#r Leu Ala Asp Lys Asp
                        340
# 345
# 350
aaa ggc att cag aaa tat ttt gtg gac ata aa
#c atc caa aac aag aag 1104
Lys Gly Ile Gln Lys Tyr Phe Val Asp Ile As
#n Ile Gln Asn Lys Lys
                355
# 360
# 365
ctg gag tct ctc ctt cag agc atg gag atg gc
#a cac agt ggc tct ctg 1152
Leu Glu Ser Leu Leu Gln Ser Met Glu Met Al
#a His Ser Gly Ser Leu
        370
# 375
# 380
agg gac gaa ctg tgc cta gac ttt cca tgt ga
#t tcc cca gag aag agc 1200
Arg Asp Glu Leu Cys Leu Asp Phe Pro Cys As
#p Ser Pro Glu Lys Ser
385 3
#90 3
#95 4
#00
tta acc ctc aac ccc cct ctt gac aca atg gc
#a gat ggg tta tct ctg 1248
Leu Thr Leu Asn Pro Pro Leu Asp Thr Met Al
#a Asp Gly Leu Ser Leu
                                405
# 410
# 415
gaa gag cag gtc acg ggg gaa ggg gct gac ag
#g gag cta ctg gta gga 1296
Glu Glu Gln Val Thr Gly Glu Gly Ala Asp Ar
#g Glu Leu Leu Val Gly
                        420
# 425
# 430
gat agc ata gcc aac agc aca gat ttg ttc ga
#t gag ata gtg aca gcc 1344
Asp Ser Ile Ala Asn Ser Thr Asp Leu Phe As
#p Glu Ile Val Thr Ala
                435
# 440
# 445
acc acc aca gaa tct ggt gac ctg gag ctt gt
#g cat tcc acc cct ggg 1392
Thr Thr Thr Glu Ser Gly Asp Leu Glu Leu Va
#l His Ser Thr Pro Gly
        450
# 455
# 460
gct aac gtc ctg gag ctg ctg ccc ata gtc at
#g ggt cag gag gag ggc 1440
Ala Asn Val Leu Glu Leu Leu Pro Ile Val Me
#t Gly Gln Glu Glu Gly
465 4
#70 4
#75 4
#80
agt gtg gtg gtg gag cga gcc gtt cag acc ga
#c gtg gtg ccc tac agc 1488
Ser Val Val Val Glu Arg Ala Val Gln Thr As
#p Val Val Pro Tyr Ser
                                485
# 490
# 495
cca gcc atc tca gag ctc att cag agt gtg ct
#g cag aag ctc cag gac 1536
Pro Ala Ile Ser Glu Leu Ile Gln Ser Val Le
#u Gln Lys Leu Gln Asp
                        500
# 505
# 510
ccc tgt ccc tcg agc ttg gcg tcc cct gat ga
#g tct gaa cca gac tcg 1584
Pro Cys Pro Ser Ser Leu Ala Ser Pro Asp Gl
#u Ser Glu Pro Asp Ser
                515
# 520
# 525
atg gag agc ttc cca gag tcc ctc tct gcc tt
#a gtg gtt gat tta act 1632
Met Glu Ser Phe Pro Glu Ser Leu Ser Ala Le
#u Val Val Asp Leu Thr
        530
# 535
# 540
cca aga aat cca aac tca gcc atc ctt ttg tc
#t ccc gtg gag acc ccc 1680
Pro Arg Asn Pro Asn Ser Ala Ile Leu Leu Se
#r Pro Val Glu Thr Pro
545 5
#50 5
#55 5
#60
tac gcc aat gtg gat gca gaa gtt cat gca aa
#c cgc ctc atg aga gag 1728
Tyr Ala Asn Val Asp Ala Glu Val His Ala As
#n Arg Leu Met Arg Glu
                                565
# 570
# 575
ctg gat ttt gca gcc tgc gtg gaa gag agg tt
#g gat ggt gtc atc cca 1776
Leu Asp Phe Ala Ala Cys Val Glu Glu Arg Le
#u Asp Gly Val Ile Pro
                        580
# 585
# 590
ctg gct cgc ggg ggc gtc gtg agg cag tac tg
#g agc agc agc ttc ctg 1824
Leu Ala Arg Gly Gly Val Val Arg Gln Tyr Tr
#p Ser Ser Ser Phe Leu
                595
# 600
# 605
gtg gat ctc ctg gct gtg gct gcc ccc gtg gt
#c ccc acg gtt ctg tgg 1872
Val Asp Leu Leu Ala Val Ala Ala Pro Val Va
#l Pro Thr Val Leu Trp
        610
# 615
# 620
gca ttc agt act cag aga ggg gga acg gat cc
#t gtg tat aac atc ggg 1920
Ala Phe Ser Thr Gln Arg Gly Gly Thr Asp Pr
#o Val Tyr Asn Ile Gly
625 6
#30 6
#35 6
#40
gcc ttg ctc agg ggc tgt tgc gtg gtt gcc ct
#g cat tcg ctc cgc cgc 1968
Ala Leu Leu Arg Gly Cys Cys Val Val Ala Le
#u His Ser Leu Arg Arg
                                645
# 650
# 655
acc gcc ttc cgt atc aaa acc taa
#
# 1992
Thr Ala Phe Arg Ile Lys Thr
                        660
<210> SEQ ID NO 4
<211> LENGTH: 663
<212> TYPE: PRT
<213> ORGANISM: Homo sapiens
<400> SEQUENCE: 4
Met Gly Pro Leu Arg Glu Ser Lys Lys Glu Hi
#s Arg Val Gln His His
    1 5
# 10
# 15
Asp Lys Glu Ile Ser Arg Ser Arg Ile Pro Ar
#g Leu Ile Leu Arg Pro
                          20
# 25
# 30
His Met Pro Gln Gln Gln His Lys Val Ser Pr
#o Ala Ser Glu Ser Pro
                  35
# 40
# 45
Phe Ser Glu Glu Glu Ser Arg Glu Phe Asn Pr
#o Ser Ser Ser Gly Arg
          50
# 55
# 60
Ser Ala Arg Thr Val Ser Ser Asn Ser Phe Cy
#s Ser Asp Asp Thr Gly
  65
# 70
# 75
# 80
Cys Pro Ser Ser Gln Ser Val Ser Pro Val Ly
#s Thr Pro Ser Asp Ala
                                  85
# 90
# 95
Gly Asn Ser Pro Ile Gly Phe Cys Pro Gly Se
#r Asp Glu Gly Phe Thr
                        100
# 105
# 110
Arg Lys Lys Cys Thr Ile Gly Met Val Gly Gl
#u Gly Ser Ile Gln Ser
                115
# 120
# 125
Ser Arg Tyr Lys Lys Glu Ser Lys Ser Gly Le
#u Val Lys Pro Gly Ser
        130
# 135
# 140
Glu Ala Asp Phe Ser Ser Ser Ser Ser Thr Gl
#y Ser Ile Ser Ala Pro
145 1
#50 1
#55 1
#60
Glu Val His Met Ser Thr Ala Gly Ser Lys Ar
#g Ser Ser Ser Ser Arg
                                165
# 170
# 175
Asn Arg Gly Pro His Gly Arg Ser Asn Gly Al
#a Ser Ser His Lys Pro
                        180
# 185
# 190
Gly Ser Ser Pro Ser Ser Pro Arg Glu Lys As
#p Leu Leu Ser Met Leu
                195
# 200
# 205
Cys Arg Asn Gln Leu Ser Pro Val Asn Ile Hi
#s Pro Ser Tyr Ala Pro
        210
# 215
# 220
Ser Ser Pro Ser Ser Ser Asn Ser Gly Ser Ty
#r Lys Gly Ser Asp Cys
225 2
#30 2
#35 2
#40
Ser Pro Ile Met Arg Arg Ser Gly Arg Tyr Me
#t Ser Cys Gly Glu Asn
                                245
# 250
# 255
His Gly Val Arg Pro Pro Asn Pro Glu Gln Ty
#r Leu Thr Pro Leu Gln
                        260
# 265
# 270
Gln Lys Glu Val Thr Val Arg His Leu Lys Th
#r Lys Leu Lys Glu Ser
                275
# 280
# 285
Glu Arg Arg Leu His Glu Arg Glu Ser Glu Il
#e Val Glu Leu Lys Ser
        290
# 295
# 300
Gln Leu Ala Arg Met Arg Glu Asp Trp Ile Gl
#u Glu Glu Cys His Arg
305 3
#10 3
#15 3
#20
Val Glu Ala Gln Leu Ala Leu Lys Glu Ala Ar
#g Lys Glu Ile Lys Gln
                                325
# 330
# 335
Leu Lys Gln Val Ile Glu Thr Met Arg Ser Se
#r Leu Ala Asp Lys Asp
                        340
# 345
# 350
Lys Gly Ile Gln Lys Tyr Phe Val Asp Ile As
#n Ile Gln Asn Lys Lys
                355
# 360
# 365
Leu Glu Ser Leu Leu Gln Ser Met Glu Met Al
#a His Ser Gly Ser Leu
        370
# 375
# 380
Arg Asp Glu Leu Cys Leu Asp Phe Pro Cys As
#p Ser Pro Glu Lys Ser
385 3
#90 3
#95 4
#00
Leu Thr Leu Asn Pro Pro Leu Asp Thr Met Al
#a Asp Gly Leu Ser Leu
                                405
# 410
# 415
Glu Glu Gln Val Thr Gly Glu Gly Ala Asp Ar
#g Glu Leu Leu Val Gly
                        420
# 425
# 430
Asp Ser Ile Ala Asn Ser Thr Asp Leu Phe As
#p Glu Ile Val Thr Ala
                435
# 440
# 445
Thr Thr Thr Glu Ser Gly Asp Leu Glu Leu Va
#l His Ser Thr Pro Gly
        450
# 455
# 460
Ala Asn Val Leu Glu Leu Leu Pro Ile Val Me
#t Gly Gln Glu Glu Gly
465 4
#70 4
#75 4
#80
Ser Val Val Val Glu Arg Ala Val Gln Thr As
#p Val Val Pro Tyr Ser
                                485
# 490
# 495
Pro Ala Ile Ser Glu Leu Ile Gln Ser Val Le
#u Gln Lys Leu Gln Asp
                        500
# 505
# 510
Pro Cys Pro Ser Ser Leu Ala Ser Pro Asp Gl
#u Ser Glu Pro Asp Ser
                515
# 520
# 525
Met Glu Ser Phe Pro Glu Ser Leu Ser Ala Le
#u Val Val Asp Leu Thr
        530
# 535
# 540
Pro Arg Asn Pro Asn Ser Ala Ile Leu Leu Se
#r Pro Val Glu Thr Pro
545 5
#50 5
#55 5
#60
Tyr Ala Asn Val Asp Ala Glu Val His Ala As
#n Arg Leu Met Arg Glu
                                565
# 570
# 575
Leu Asp Phe Ala Ala Cys Val Glu Glu Arg Le
#u Asp Gly Val Ile Pro
                        580
# 585
# 590
Leu Ala Arg Gly Gly Val Val Arg Gln Tyr Tr
#p Ser Ser Ser Phe Leu
                595
# 600
# 605
Val Asp Leu Leu Ala Val Ala Ala Pro Val Va
#l Pro Thr Val Leu Trp
        610
# 615
# 620
Ala Phe Ser Thr Gln Arg Gly Gly Thr Asp Pr
#o Val Tyr Asn Ile Gly
625 6
#30 6
#35 6
#40
Ala Leu Leu Arg Gly Cys Cys Val Val Ala Le
#u His Ser Leu Arg Arg
                                645
# 650
# 655
Thr Ala Phe Arg Ile Lys Thr
                        660
<210> SEQ ID NO 5
<211> LENGTH: 477
<212> TYPE: DNA
<213> ORGANISM: Rattus norvegicus
<400> SEQUENCE: 5
ggctcctaca aaggaagcga ctgtagtcca gtcatgagga ggtctggacg at
#atatgtct 60
tgtggagaaa atcatggcgt caaaccccca aatccagaac agtatttgac ac
#ctctgcag 120
cagaaggagg tcacagtgag gcatttgagg accaagctga aggagtctga gc
#gccgactc 180
catgagaggg aatctgaaat catggagctc aagtctcagc tggctcggat ga
#gggaagac 240
tggatagagg aagagtgcca cagggtggag gctcagttgg cgctcaaaga ag
#ccagaaaa 300
gagattaagc agctcaaaca ggtcattgag actatgagga gcagcttggc tg
#ataaagat 360
aaaggcattc agaagtactt tgtggacata aacatccaaa acaagaaact gg
#agtctctg 420
cttcaaagca tggagatggc gcacaatagt tccctgaggg atgaactgtg tc
#tagac 477
<210> SEQ ID NO 6
<211> LENGTH: 159
<212> TYPE: PRT
<213> ORGANISM: Rattus norvegicus
<400> SEQUENCE: 6
Gly Ser Tyr Lys Gly Ser Asp Cys Ser Pro Va
#l Met Arg Arg Ser Gly
    1 5
# 10
# 15
Arg Tyr Met Ser Cys Gly Glu Asn His Gly Va
#l Lys Pro Pro Asn Pro
                          20
# 25
# 30
Glu Gln Tyr Leu Thr Pro Leu Gln Gln Lys Gl
#u Val Thr Val Arg His
                  35
# 40
# 45
Leu Arg Thr Lys Leu Lys Glu Ser Glu Arg Ar
#g Leu His Glu Arg Glu
          50
# 55
# 60
Ser Glu Ile Met Glu Leu Lys Ser Gln Leu Al
#a Arg Met Arg Glu Asp
  65
# 70
# 75
# 80
Trp Ile Glu Glu Glu Cys His Arg Val Glu Al
#a Gln Leu Ala Leu Lys
                                  85
# 90
# 95
Glu Ala Arg Lys Glu Ile Lys Gln Leu Lys Gl
#n Val Ile Glu Thr Met
                        100
# 105
# 110
Arg Ser Ser Leu Ala Asp Lys Asp Lys Gly Il
#e Gln Lys Tyr Phe Val
                115
# 120
# 125
Asp Ile Asn Ile Gln Asn Lys Lys Leu Glu Se
#r Leu Leu Gln Ser Met
        130
# 135
# 140
Glu Met Ala His Asn Ser Ser Leu Arg Asp Gl
#u Leu Cys Leu Asp
145 1
#50 1
#55
<210> SEQ ID NO 7
<211> LENGTH: 477
<212> TYPE: DNA
<213> ORGANISM: Homo sapiens
<400> SEQUENCE: 7
ggctcctaca aaggaagcga ctgtagcccc atcatgaggc gttctggaag gt
#acatgtct 60
tgcggtgaaa atcatggtgt cagaccccca aacccagagc agtatttgac tc
#cactgcag 120
cagaaagagg tgacagtgag acacctcaaa accaagctga aggaatctga gc
#gccgactc 180
catgaaaggg aaagtgaaat cgtggagctt aagtcccagc tggcccgcat gc
#gagaggac 240
tggattgagg aggagtgtca ccgggtagag gcccagttgg cactcaaaga ag
#ccaggaaa 300
gagattaaac agctcaaaca ggtcatcgaa accatgcgga gcagcttggc tg
#ataaagat 360
aaaggcattc agaaatattt tgtggacata aacatccaaa acaagaagct gg
#agtctctc 420
cttcagagca tggagatggc acacagtggc tctctgaggg acgaactgtg cc
#tagac 477
<210> SEQ ID NO 8
<211> LENGTH: 159
<212> TYPE: PRT
<213> ORGANISM: Homo sapiens
<400> SEQUENCE: 8
Gly Ser Tyr Lys Gly Ser Asp Cys Ser Pro Il
#e Met Arg Arg Ser Gly
    1 5
# 10
# 15
Arg Tyr Met Ser Cys Gly Glu Asn His Gly Va
#l Arg Pro Pro Asn Pro
                          20
# 25
# 30
Glu Gln Tyr Leu Thr Pro Leu Gln Gln Lys Gl
#u Val Thr Val Arg His
                  35
# 40
# 45
Leu Lys Thr Lys Leu Lys Glu Ser Glu Arg Ar
#g Leu His Glu Arg Glu
          50
# 55
# 60
Ser Glu Ile Val Glu Leu Lys Ser Gln Leu Al
#a Arg Met Arg Glu Asp
  65
# 70
# 75
# 80
Trp Ile Glu Glu Glu Cys His Arg Val Glu Al
#a Gln Leu Ala Leu Lys
                                  85
# 90
# 95
Glu Ala Arg Lys Glu Ile Lys Gln Leu Lys Gl
#n Val Ile Glu Thr Met
                        100
# 105
# 110
Arg Ser Ser Leu Ala Asp Lys Asp Lys Gly Il
#e Gln Lys Tyr Phe Val
                115
# 120
# 125
Asp Ile Asn Ile Gln Asn Lys Lys Leu Glu Se
#r Leu Leu Gln Ser Met
        130
# 135
# 140
Glu Met Ala His Ser Gly Ser Leu Arg Asp Gl
#u Leu Cys Leu Asp
145 1
#50 1
#55
<210> SEQ ID NO 9
<211> LENGTH: 30
<212> TYPE: DNA
<213> ORGANISM: Artificial Sequence
<220> FEATURE:
<223> OTHER INFORMATION: primer
<400> SEQUENCE: 9
acagtcgact atgtctgcac tccgaaggaa
#
# 30
<210> SEQ ID NO 10
<211> LENGTH: 35
<212> TYPE: DNA
<213> ORGANISM: Artificial Sequence
<220> FEATURE:
<223> OTHER INFORMATION: primer
<400> SEQUENCE: 10
accgctggag cccgaagaga taaagaggtt ccaac
#
# 35
<210> SEQ ID NO 11
<211> LENGTH: 36
<212> TYPE: DNA
<213> ORGANISM: Artificial Sequence
<220> FEATURE:
<223> OTHER INFORMATION: primer
<400> SEQUENCE: 11
tcttcgggct ccagcggtat caagatgtcc cagccc
#
# 36
<210> SEQ ID NO 12
<211> LENGTH: 30
<212> TYPE: DNA
<213> ORGANISM: Artificial Sequence
<220> FEATURE:
<223> OTHER INFORMATION: primer
<400> SEQUENCE: 12
gtcactagtg gtgttttgct atgtgaagcg
#
# 30
<210> SEQ ID NO 13
<211> LENGTH: 22
<212> TYPE: DNA
<213> ORGANISM: Artificial Sequence
<220> FEATURE:
<223> OTHER INFORMATION: primer
<400> SEQUENCE: 13
tcgcacagct gataggatta gg
#
# 22
<210> SEQ ID NO 14
<211> LENGTH: 22
<212> TYPE: DNA
<213> ORGANISM: Artificial Sequence
<220> FEATURE:
<223> OTHER INFORMATION: primer
<400> SEQUENCE: 14
cctcatgatg gggctacagt cg
#
# 22
<210> SEQ ID NO 15
<211> LENGTH: 21
<212> TYPE: DNA
<213> ORGANISM: Artificial Sequence
<220> FEATURE:
<223> OTHER INFORMATION: primer
<400> SEQUENCE: 15
tttgtcagcc ctgattgagc c
#
#
#21
<210> SEQ ID NO 16
<211> LENGTH: 22
<212> TYPE: DNA
<213> ORGANISM: Artificial Sequence
<220> FEATURE:
<223> OTHER INFORMATION: primer
<400> SEQUENCE: 16
gagctgctgg ggttgaactc tc
#
# 22
<210> SEQ ID NO 17
<211> LENGTH: 22
<212> TYPE: DNA
<213> ORGANISM: Artificial Sequence
<220> FEATURE:
<223> OTHER INFORMATION: primer
<400> SEQUENCE: 17
gagagttcaa ccccagcagc tc
#
# 22
<210> SEQ ID NO 18
<211> LENGTH: 22
<212> TYPE: DNA
<213> ORGANISM: Artificial Sequence
<220> FEATURE:
<223> OTHER INFORMATION: primer
<400> SEQUENCE: 18
cctcatgatg gggctacagt cg
#
# 22
<210> SEQ ID NO 19
<211> LENGTH: 18
<212> TYPE: DNA
<213> ORGANISM: Artificial Sequence
<220> FEATURE:
<223> OTHER INFORMATION: primer
<400> SEQUENCE: 19
gagagcaagg agcacaga
#
#
# 18
<210> SEQ ID NO 20
<211> LENGTH: 22
<212> TYPE: DNA
<213> ORGANISM: Artificial Sequence
<220> FEATURE:
<223> OTHER INFORMATION: primer
<400> SEQUENCE: 20
cctcatgatg gggctacagt cg
#
# 22
<210> SEQ ID NO 21
<211> LENGTH: 31
<212> TYPE: DNA
<213> ORGANISM: Artificial Sequence
<220> FEATURE:
<223> OTHER INFORMATION: primer
<400> SEQUENCE: 21
taggtcgacc atgtttagcg agcatgcggt t
#
# 31
<210> SEQ ID NO 22
<211> LENGTH: 31
<212> TYPE: DNA
<213> ORGANISM: Artificial Sequence
<220> FEATURE:
<223> OTHER INFORMATION: primer
<400> SEQUENCE: 22
gtcactagtt ggggtgtttt gctatgtgaa g
#
# 31
<210> SEQ ID NO 23
<211> LENGTH: 30
<212> TYPE: DNA
<213> ORGANISM: Artificial Sequence
<220> FEATURE:
<223> OTHER INFORMATION: primer
<400> SEQUENCE: 23
acagtcgact atgtctgcac tccgaaggaa
#
# 30
<210> SEQ ID NO 24
<211> LENGTH: 26
<212> TYPE: DNA
<213> ORGANISM: Artificial Sequence
<220> FEATURE:
<223> OTHER INFORMATION: primer
<400> SEQUENCE: 24
gtcactagtg ataaagaggt tccaac
#
# 26
<210> SEQ ID NO 25
<211> LENGTH: 25
<212> TYPE: DNA
<213> ORGANISM: Artificial Sequence
<220> FEATURE:
<223> OTHER INFORMATION: primer
<400> SEQUENCE: 25
acagtcgacg gagtctgagc gccga
#
# 25
<210> SEQ ID NO 26
<211> LENGTH: 28
<212> TYPE: DNA
<213> ORGANISM: Artificial Sequence
<220> FEATURE:
<223> OTHER INFORMATION: primer
<400> SEQUENCE: 26
gtcgcggccg cctgctcctc atagtctc
#
# 28
<210> SEQ ID NO 27
<211> LENGTH: 31
<212> TYPE: DNA
<213> ORGANISM: Artificial Sequence
<220> FEATURE:
<223> OTHER INFORMATION: primer
<400> SEQUENCE: 27
gacgtcgaca ggctcctaca aaggaagcga c
#
# 31
<210> SEQ ID NO 28
<211> LENGTH: 35
<212> TYPE: DNA
<213> ORGANISM: Artificial Sequence
<220> FEATURE:
<223> OTHER INFORMATION: primer
<400> SEQUENCE: 28
gagcggccgc tcagtctaga cacagttcat ccctc
#
# 35
<210> SEQ ID NO 29
<211> LENGTH: 31
<212> TYPE: DNA
<213> ORGANISM: Artificial Sequence
<220> FEATURE:
<223> OTHER INFORMATION: primer
<400> SEQUENCE: 29
gacgtcgaca ggctcctaca agggcagtga c
#
# 31
<210> SEQ ID NO 30
<211> LENGTH: 34
<212> TYPE: DNA
<213> ORGANISM: Artificial Sequence
<220> FEATURE:
<223> OTHER INFORMATION: primer
<400> SEQUENCE: 30
gagcggccgc tcactcccca gtgccatcct cctt
#
# 34
<210> SEQ ID NO 31
<211> LENGTH: 26
<212> TYPE: DNA
<213> ORGANISM: Artificial Sequence
<220> FEATURE:
<223> OTHER INFORMATION: primer
<400> SEQUENCE: 31
cggaattcgc aggcaacgac gagatg
#
# 26
<210> SEQ ID NO 32
<211> LENGTH: 28
<212> TYPE: DNA
<213> ORGANISM: Artificial Sequence
<220> FEATURE:
<223> OTHER INFORMATION: primer
<400> SEQUENCE: 32
cgcgtcgacg tctagacaca tgtcatcc
#
# 28
<210> SEQ ID NO 33
<211> LENGTH: 32
<212> TYPE: DNA
<213> ORGANISM: Artificial Sequence
<220> FEATURE:
<223> OTHER INFORMATION: primer
<400> SEQUENCE: 33
ctattcgatg atgaagatac cccaccaaac cc
#
# 32
<210> SEQ ID NO 34
<211> LENGTH: 31
<212> TYPE: DNA
<213> ORGANISM: Artificial Sequence
<220> FEATURE:
<223> OTHER INFORMATION: primer
<400> SEQUENCE: 34
gtgaacttgc ggggtttttc agtatctacg a
#
# 31
```

We claim:

1. An isolated nucleic acid sequence selected from the group:
a) of a nucleic acid sequence having the sequence depicted in SEQ ID NO: 1, SEQ ID NO: 3; SEQ ID NO: 5 or SEQ ID NO: 7,
b) nucleic acid sequences which are derived as a result of the degeneracy of the genetic code from the nucleic acid sequence depicted in SEQ ID NO: 1, SEQ ID NO: 3; SEQ ID NO: 5 or SEQ ID NO: 7,
c) derivatives of the nucleic acid sequence depicted in SEQ ID NO: 1 or SEQ ID NO: 3, which code for polypeptides having the amino acid sequences depicted in SEQ ID NO: 2 or SEQ ID NO: 4 and have at least 60% homology at the amino acid level, with negligible reduction in the biological activity of the polypeptides,
d) equivalents of the sequences specified under (a) to (c) which still have biological activity.

2. A protein encoded by a nucleic acid sequence as claimed in claim 1.

3. A protein complex comprising at least one protein as claimed in claim 2 and at least one other protein, where at least one of the essential biological properties of the protein depicted in SEQ ID NO: 2 or SEQ ID NO: 4, or of the protein complex, is still retained.

4. A protein complex as claimed in claim 3, where the other protein is a GIRK protein.

5. A recombinant nucleic acid construct comprising a nucleic acid sequence as claimed in claim 1 or a nucleic acid sequence as claimed in claim 1 and a sequence which codes for another protein, functionally linked to at least one genetic regulatory element.

6. A host organism transformed with a nucleic acid sequence as claimed in claim 1 or a recombinant nucleic acid construct as claimed in claim 5.

7. A transgenic animal comprising a functional or nonfunctional nucleic acid sequence as claimed in claim 1 or a functional or nonfunctional nucleic acid construct as claimed in claim 5.

8. A transgenic animal in whose germ cells or all or a part of the somatic cells, or in whose germ cells and all or a part of the somatic cells the nucleotide sequence as claimed in claim 1 has been modified by genetic engineering methods or interrupted by insertion of DNA elements.

9. The use of a nucleic acid sequence as claimed in claim 1, of a nucleic acid construct as claimed in claim 5, of a protein complex as claimed in claim 3 or protein as claimed in claim 2 for identifying proteins which show specific binding affinities for a protein complex as claimed in claim 3 or a protein as claimed in claim 2, or for identifying nucleic acids which code for proteins which show specific binding affinities for a protein complex as claimed in claim 3 or a protein as claimed in claim 2.

10. The use of the two-hybrid system or biochemical methods for identifying the interaction domains of Kirs, GIRKs with proteins as claimed in claim 2 and the use for pharmacotherapeutic intervention.

11. The use of the information resulting from an elucidation of the structure of a protein complex as claimed in claim 3 or of a protein as claimed in claim 2 for the targeted discovery or targeted production of substances with specific binding activity for a protein complex as claimed in claim 3 or a protein as claimed in claim 2.

12. The use of a protein complex as claimed in claim 3 or of a protein as claimed in claim 2 or peptide fragments thereof as antigen for generating specific mono- or polyclonal antibodies or antibody mixtures directed against proteins as claimed in claim 2 or against protein complex as claimed in claim 3.

13. A mono- or polyclonal antibody or antibody mixture which specifically recognizes proteins as claimed in claim 2 or protein complexes as claimed in claim 3.

14. The use of a nucleic acid sequence as claimed in claim 1 or of a fragment thereof for isolating a genomic sequence by homology screening, as marker for human genetic diseases or for gene therapy.

15. A method for discovering substances with specific binding affinity for a protein as claimed in claim 2, which comprises the following steps:
a) incubation of the protein as claimed in claim 2 with the substance to be tested;
b) detection of the binding to the protein of the substance to be tested.

16. A method as claimed in claim 15, wherein the detection of the binding takes place by measuring the K⁺ conductivity of GIRK proteins or the transmitter release.

17. A method for the qualitative or quantitative detection of a nucleic acid as claimed in claim 1 in a biological sample, which comprises one or more of the following steps:
a) incubation of a biological sample with a known amount of nucleic acid as claimed in claim 1 or a known amount of oligonucleotides which are suitable as primers for amplification of the nucleic acid as claimed in claim 1, or mixtures thereof,
b) detection of the nucleic acid as claimed in claim 1 by specific hybridization or PCR amplification,
c) comparison of the amount of hybridizing nucleic acid as claimed in claim 1, or of nucleic acid as claimed in claim 1 obtained by PCR amplification, with a standard.

18. A method for the qualitative and quantitative detection of a protein as claimed in claim 2 or of a protein complex as claimed in claim 3 in a biological sample, which comprises one or more of the following steps:
a) incubation of a biological sample with an antibody as claimed in claim 13 which is specifically directed against proteins as claimed in claim 2 or a protein complex as claimed in claim 3,
b) detection of the antibody/antigen complex,
c) comparison of the amounts of the antibody/antigen complex with a quantity standard.

19. A method for discovering substances which specifically bind to a protein having an amino acid sequence as claimed in claim 2, which comprises one or more of the following steps:
a) expression of the protein in eukaryotic or prokaryotic cells,
b) incubation of the protein with the substances to be tested,
c) detection of the binding of a substance to the protein.

20. A method for discovering substances which inhibit or enhance the interaction of proteins having amino acid sequences as claimed in claim 2 with interacting proteins.

21. A method as claimed in claim 20, where the interacting protein is a Kir protein.

22. A method as claimed in claim 20, where the interacting protein is a SNARE complex protein or a protein associated therewith.

23. A method as claimed in any of claims 20 to 22, where the method also includes the steps of the method as claimed in claim 15, 16 and/or 19.

24. A method as claimed in any of claims 20, 22 and 23, where the substances enhance or diminish transmitter release.

25. A drug product which comprises the nucleic acid sequence, the protein, the antibody, the protein complex of one of the preceding claims, an antisense molecule to the nucleic acid sequence of claim 1, or a substance which has been discovered in accordance with one of the preceding claims and, optionally, a pharmaceutically suitable carrier.

26. A method for detecting a disorder comprising the steps of the method as claimed in claim 17 or 18, where the standard has been selected such that it represents the expression of a healthy organism.

27. A means for diagnosing genotypes, comprising the nucleic acid as claimed in claim 1, a fragment thereof, or an antisense nucleic acid molecule thereof.

28. A method for producing a drug product, comprising the steps of one of the methods as claimed in claim 15, 16 or 17, and formulation of the discovered substance with a pharmaceutically suitable carrier.

29. The use of the nucleic acid sequence, of the protein, of the antibody as claimed in any of the preceding claims or of an antisense molecule against the nucleic acid sequence as claimed in claim 1 or of one of the substances discovered by the preceding methods, for producing a drug product for the treatment of neurological disorders.
